(19) United States
(12) Patent Application Publication
(10) Pub. No.: US 2003/0148519 A1
(43) Pub. Date: Aug. 7, 2003

(54) Intracellular expression and delivery of siRNAs in mammalian cells

(76) Inventors: David R. Engelke, Ann Arbor, MI (US); Paul D. Good, Grass Lake, MI (US); Cynthia P. Paul, Northville, MI (US)

Correspondence Address: Jaen Andrews, MEDLEN & CARROLL, LLP, Suite 350, 101 Howard Street, San Francisco, CA, 94105, US

(21) Appl. No.: 10/294,957

(22) Filed: Nov. 14, 2002

Related U.S. Application Data
(60) Provisional application No. 60/332,170, filed on Nov. 14, 2001.

(51) Int. Cl. C12N 15/85; C12N 5/06

(52) U.S. Cl. 435/455; 435/325; 435/320.1

(57) ABSTRACT

The present invention relates to compositions and methods for intracellular expression and delivery of siRNAs in mammalian cells. The siRNA is transcribed intracellularly as a double stranded RNA of about 18 to about 25 base pairs long from an expression cassette. Intracellular expression of siRNA is effective at reducing or eliminating expression of the targeted genes, and is applicable to reverse genetic analysis of genes and to genetic therapy, as for example to inhibiting expression of pathogenic genes and oncogenes.

The present application claims priority from provisional patent application Serial No. 60/332,170, filed Nov. 14, 2001.

The present application was funded in part with government support under grant number NIH AI40936. The government may have certain rights in this application.

### FIELD OF THE INVENTION

The present invention relates to gene silencing, and in particular to compositions and methods for intracellular expression and delivery of siRNAs in mammalian cells.

### BACKGROUND OF THE INVENTION

Several kinds of potential nucleic acid therapeutics have been explored over the last two decades, including RNA inhibitors such as antisense, ribozymes (catalytic RNAs), and artificial ligand inhibitors (“aptamers”). These therapeutics are designed to silence gene expression, and thus to alleviate the effects of undesirable genes, be they endogenous to an organism or exogenous, such as bacterial or viral in origin. Because it is difficult to apply these to cells externally, there has been significant interest in expressing them within cells. However, expression of these therapeutics intracellularly has proved quite difficult as well; this difficulty is thought to be due to several factors. These include, for RNA-based therapeutics as an example, the considerations of finding their targets, folding into the effective configuration, and possibly interacting with the appropriate proteins while avoiding interactions with inappropriate proteins. There have been isolated promising results (see, for example, Bertrand, E et al. (1997) RNA3: 75-88; Good, P D et al. (1997) Gene Therapy4: 45-54), but no therapeutics have yet resulted.

Recently the field of reverse genetic analysis, or gene silencing, has been revolutionized by the discovery of potent, sequence specific inactivation of gene function, which can be induced by double-stranded RNA (dsRNA). In contrast to the limited effectiveness of inhibiting gene expression with antisense, ribozymes, and aptamers, it has been known for a few years that “RNA interference” (RNAi) works quite well to suppress expression of a gene's RNA in lower eukaryotes. RNAi is the use of double-stranded RNA to silence the expression of specific mRNAs, where it is believed that the targeted RNA is degraded, although this has not yet been confirmed. The active agent in RNAi is a long double-stranded (antiparallel duplex) RNA, with one of the strands corresponding or complementary to the RNA which is to be inhibited. The inhibited RNA is the target RNA. The long double stranded RNA is chopped into smaller duplexes of approximately 20 to 25 nucleotide pairs, after which the mechanism by which the smaller RNAs inhibit expression of the target is largely unknown at this time. For mammalian cells, however, it was thought that RNAi might be suitable only for studies on the oocyte and the preimplantation embryo. In mammalian cells other than these, longer RNA duplexes provoke different responses.

In this response, termed sequence non-specific RNA interference, dsRNA triggers a non-specific inhibition of protein synthesis that overwhelms any sequence-specific RNAi effect. This effect is induced by dsRNA of greater than about 30 base pairs, and appears to be due to an interferon response. The interferon response is thought to be initiated by dsRNA of greater than about 30 base pairs, where the dsRNA binds and activates the protein PKR and 2′,5′-oligonucleotide synthetase (2′,5′-AS). Activated PKR stalls translation by phosphorylation of the translation initiation factors eIF2alpha, and activated 2′,5′-AS causes mRNA degradation by 2′,5′-oligonucleotide-activated ribonuclease L. These responses are intrinsically sequence-nonspecific to the inducing dsRNA; they also frequently result in apoptosis, or cell death. Thus, most somatic mammalian cells undergo apoptosis when exposed to the concentrations of dsRNA that induce RNAi in lower eukaryotic cells.

Recently, though, it was shown that RNAi would work in human cells if the RNA strands were provided as pre-sized duplexes of about 19 nucleotide pairs, and RNAi worked particularly well with small unpaired 3′ extensions on the end of each strand (Elbashir et al. (2001) Nature 411: 494-498). In this report, “short interfering RNA” (siRNA, also referred to as small interfering RNA) were applied to cultured cells by transfection in oligofectamine micelles. These RNA duplexes are too short to elicit sequence-nonspecific responses like apoptosis, yet they efficiently initiate RNAi. This was a stunning discovery and many laboratories around the country immediately rushed to have siRNA made to knock out their favorite gene in mammalian cells. The results demonstrate that siRNA appears to work quite well in most instances, far better and more consistently than do ribozymes, antisense or other nucleic acid agents. However, a major limitation to the use of siRNA in mammalian cells is the method of delivery.

Currently, the synthesis of the siRNA is expensive. Moreover, inducing cells to take up exogenous nucleic acids is a short-term treatment and is very difficult to achieve in some cultured cell types. This methodology does not permit long-term expression of the siRNA in cells or use of siRNA in tissues, organs, and whole organisms. It had also not been demonstrated that siRNA could effectively be expressed from recombinant DNA constructs to suppress expression of a target gene. Thus, what is needed are methods to express and deliver siRNA intracellularly in mammalian cells, and indeed in other cells as well.

### SUMMARY OF THE INVENTION

It is therefore an object of the present invention to provide compositions and methods for expression of siRNA in an animal cell.

Therefore, the present invention provides a composition comprising an siRNA molecule, wherein the molecule comprises a first region, a second region, and a third region, wherein the first region is complementary to and paired to the second region forming a double stranded region such that the double stranded region comprises about 18 to about 25 nucleotide pairs, and wherein the third region links said first region to said second region. In some embodiments, the third region is a short loop sequence, from about four to about ten nucleotides in length. In other embodiments, the third region is a part to all of a tRNA sequence; in some embodiments, the tRNA sequence is modified by additions, deletions, or substitutions of nucleotides.

The present invention also provides a composition as described above, wherein the first region or the second region of the siRNA molecule is complementary to a region of a target RNA molecule.

The present invention also provides any of the compositions described above, wherein the siRNA molecule further comprises at least a fourth region, wherein the fourth region enhances the function of the siRNA. In some embodiments, the fourth regions is a 3′ end nucleotide overhang; in some preferred embodiments, such nucleotide overhangs are about one to four nucleotides in length. In other preferred embodiments, the antisense strand has a 3′ nucleotide overhang. In any of these embodiments, the fourth region may comprise a part to all of at least one of the first, the second, and the third region.

The present invention also provides any of the compositions described above, wherein the fourth region of the siRNA comprises a cellular destination signal. In some embodiments, the destination signal directs the siRNA to the cytoplasm. In other embodiments, the destination signal directs the siRNA to the nucleolus. In any of these embodiments, the fourth region may comprise a part to all of at least one of the first, the second, and the third region.

In other embodiments, the present invention provides an expression cassette comprising a promoter operably linked to a gene encoding an siRNA molecule, wherein the siRNA molecule comprises a first region and a second region, wherein the first region is complementary to and paired to the second region forming a double stranded region such that the double stranded region comprises about 18 to about 25 nucleotide pairs.

In yet other embodiments, the present invention provides an expression cassette as described above, wherein the siRNA molecule further comprises a third region, wherein the third region links the first region to the second region.

In yet other embodiments, the present invention provides any of the expression cassettes as described above, comprising a sequence encoding any of the siRNA molecules described above.

In yet other embodiments, the present invention provides any of the expression cassettes as described above, wherein the promoter is a polymerase III gene promoter. In other embodiments, the promoter is from a nuclear or a ribosomal RNA gene. In other embodiments, the promoter is all or a part of the polymerase III gene promoter or the promoter from a nuclear or a ribosomal RNA gene. In other embodiments, the expression cassette further comprises at least some additional sequences from the coding sequence for a nuclear or a ribosomal RNA gene. In other embodiments, the promoter comprises a cellular destination signal; in some of these embodiments, the destination signal in the transcriptional promoter of the RNA directs the retention of the siRNA in the nucleus. In some preferred embodiments, the expression cassette comprises a promoter from a U6 snRNA gene, from a 5S rRNA gene, or from a 7SL signal recognition particle RNA gene. In other preferred embodiments, the expression cassette comprises a promoter from a U6 snRNA gene, and from one to twenty seven of the first nucleotides of the coding sequence of the U6 snRNA gene.

The present invention also provides a vector comprising at least one of any of the expression cassettes described above; preferably, the vector is an animal cell vector, and most preferably the vector is a mammalian cell vector. In other embodiments, the vector further comprises at least one additional second gene operably linked to a second promoter; in some further embodiments, the second gene is selected from the group consisting of marker genes, reporter genes, and selectable genes.

The present invention also provides a mammalian cell expressing any of the siRNA molecules as described above. The present invention also provides a mammalian cell transfected with any of the expression cassettes as described above. The present invention also provides a mammalian cell transfected with any of the vectors as described above.

The present invention also provides a method of transfecting a mammalian cell, comprising providing a mammalian cell and any of the expression cassettes described above, and transfecting the cell with the cassette. The present invention also provides a method of expressing siRNA in a mammalian cell, comprising providing a mammalian cell transfected with any of the expression cassettes described above, and growing said cell under conditions such that the siRNA is expressed. The present invention also provides a method of expressing siRNA in a mammalian cell, comprising providing a mammalian cell and any of the expression cassettes described above, and transfecting the cell with the cassette under conditions such that the cassette is expressed. In other embodiments, the expression cassette is provided within a vector.

The present invention also provides a method of decreasing expression of a gene in a mammalian cell, comprising providing a cell transfected with any of the expression cassettes as described above, where the siRNA targets a gene in the cell; and growing the cell under conditions such that the cassette is expressed, thereby decreasing expression of the gene in the mammalian cell. In other embodiments, the expression cassette is provided within a vector.

The present invention also provides a method of decreasing expression of a gene in a mammalian cell, comprising providing a mammalian cell and any of the expression cassettes described above, where the siRNA targets a gene in the cell, and transfecting the cell with the cassette under conditions such that the cassette is expressed, thereby decreasing expression of the gene in the mammalian cell. In other embodiments, the expression cassette is provided within a vector.

### DESCRIPTION OF THE FIGURES

FIG. 1A shows the general structure of the human U6 snRNA gene promoter expression cassette schematically. The upper part of the Figure shows the scheme of the expression cassette, while the lower part shows the expected RNA transcripts from two different expression cassettes, U+27 and U+1, where the sequences from the U6 snRNA gene are shown, and the location of the RNA insert indicated. Note that including a sequence encoding 4 or more contiguous Us in the “RNA insert” region will create a transcription terminator before the cassette stem/terminator is reached.

FIG. 1B shows the expected structure of the anti-lamin A/C siRNA transcript from the U6+1 gene promoter expression cassette. If the transcript were expressed from the U6+27 cassette, the first 27 nucleotides of the human U6 RNA would also be expected (see FIG. 1A). Note that a UUUU sequence has deliberately been inserted at the end of the siRNA double-stranded region. In vivo this is expected to yield a mixture of molecules with 2-4 U residues due to either early pausing or exonuclease trimming of the 3′ end.

FIG. 2A shows the general structure of the human 7SL signal recognition particle RNA gene promoter expression cassette; the upper part shows a schematic of the expression cassette, while the lower portion shows the expected RNA transcript expressed from the promoter. The RNA transcript does not include the RNA insert, but the location of the transcript is indicated.

FIG. 2B shows the general structure of the human 5S ribosomal RNA gene promoter expression cassette; the upper part shows a schematic of the expression cassette, while the lower portion shows the expected RNA transcript expressed from the promoter. The RNA transcript does not include the RNA insert, but the location of the transcript is indicated.

FIG. 3 shows four expected RNA transcripts expressed from four different U6+1 RNA gene promoter expression cassettes. Each would begin immediately after the SalI sequence from the cassette (included in the figure) and most termination occurs after the UUU at the insert 3′ terminus. The genes encoding the different RNAs are indicated under each transcript; these are an anti-lamin A/C siRNA, the 5′ strand only (or sense strand) with the tetraloop of the anti-lamin A/C siRNA, the 3′ strand only (or antisense strand) with the tetraloop of the anti-lamin A/C siRNA, and a strand switched anti-lamin A/C siRNA.

FIG. 4 shows a restriction of the plasmid or vector into which an expression cassette coding an siRNA gene can be inserted.

FIG. 5 shows a nucleic acid sequence of the vector shown in FIG. 4. This particular sequence is of the vector with the U6+27 gene promoter expression cassette but without a gene encoding an siRNA gene. The cassette is between the restriction sites BamHl and PshAI (which is part of a of polylinker).

FIG. 6 shows the results of inhibiting lamin A/C expression by treatment of human HeLa cells with synthetic siRNA. The cells were treated with either synthetic siRNA (upper set of photos) or no siRNA (lower set of photos). The cells were examined for the presence of lamin A/C only (by staining with monoclonal antibody against lamin A/C, which fluoresces red, first column) or the presence of nuclear DNA (by using DAPI to visualize the DNA, which fluoresces blue, second column—this stain thus demonstrates the presence of cells). These two sets of results were then merged (third column of photos). In the absence of siRNA, all cells fluoresce red; only those cells which were transfected with synthetic siRNA, and in which endogenous lamin A/C disappeared, do not fluoresce red.

FIG. 7 shows the results of inhibiting lamin A/C expression with U6 gene promoter expression cassettes making anti-lamin siRNA intracellularly. HeLa cells were transfected with either plasmids containing either the empty U6+1 cassette (no RNA insert, bottom row), the U6+1 cassette expressing anti-lamin AC siRNA (the second row), or the U6+27 cassette expressing anti-lamin A/C siRNA (the upper row); all the cells were also transfected with a separate plasmid encoding β-galactosidase; this plasmid was a reporter for cell transfection, as cells which take up one plasmid generally take up both. The cells were stained with antibodies to lamin A/C (which fluoresces red, first column) or the activity of β-galactosidase detected (which fluoresces green, second column). The two sets of results were then merged (third column). For the control cells (empty U6+1 gene promoter expression cassette), cells were either only stained red for lamin A/C (not transfected) or stained with both green and red (indicating that the cells were transfected, but that no anti-lamin siRNA was expressed from the empty expression cassette). For the U6+1 and U6+27 gene promoter expression cassettes (both containing anti-lamin A/C siRNA genes), cells were generally either stained red (indicating that they contain nuclear lamin A/C protein) or only green (indicating that they were transfected and that lamin A/C was absent).

FIG. 8 shows the merged results from cells stained for the presence of lamin A/C (which fluoresce red) with those for cells expressing β-galactosidase (which fluoresce green) for cells transfected with expression cassettes containing anti-lamin siRNA genes or variants. These anti-lamin A/C siRNA are expressed from either the 5S rRNA gene promoter expression cassette or the 7SL rnpRNA gene promoter expression cassette. The variant siRNA genes, encoding the switched strand variant (“reverse strands”), the antisense strand only, or the sense strand only, were expressed from the U6+27 gene promoter expression cassette.

FIG. 9 shows four expected RNA transcripts expressed from four different inserts which can be inserted into the gene promoter expression cassettes shown in FIGS. 1 and 2. The inserts are a ribozyme (FIG. 9A), Rev binding element (RBE) decoy (FIG. 9B), and two hairpin siRNAs targeted against two different sequences within HIV-1 polymerase (FIGS. 9C and 9D). Sequences and expected secondary structures of the inserts are shown.

FIG. 10 shows the reduction in HIV-1 gene expression by hairpin siRNAs expressed from the U6 promoter. HIV-1 mRNA was targeted using hairpin siRNAs expressed from the U6+27 cassette. HIV-1 gene expression following cotransfection of cells with provirus and with the hairpin siRNA expression cassette was measured using an immunoassay for HIV p24. The siRNA inserts were directed against positions 2315 and 2568 in the HIV-1 sequence. Their ability to interfere with HIV-1 gene expression was compared to that of a control U6+27 cassette with no insert

### DEFINITIONS

To facilitate an understanding of the present invention, a number of terms and phrases as used herein are defined below:

The terms “protein” and “polypeptide” refer to compounds comprising amino acids joined via peptide bonds and are used interchangeably.

As used herein, where “amino acid sequence” is recited herein to refer to an amino acid sequence of a protein molecule. An “amino acid sequence” can be deduced from the nucleic acid sequence encoding the protein. However, terms such as “polypeptide” or “protein” are not meant to limit the amino acid sequence to the deduced amino acid sequence, but include post-translational modifications of the deduced amino acid sequences, such as amino acid deletions, additions, and modifications such as glycolsylations and addition of lipid moieties.

The term “portion” when used in reference to a protein (as in “a portion of a given protein”) refers to fragments of that protein. The fragments may range in size from four amino acid residues to the entire amino sequence minus one amino acid.

The term “chimera” when used in reference to a polypeptide refers to the expression product of two or more coding sequences obtained from different genes, that have been cloned together and that, after translation, act as a single polypeptide sequence. Chimeric polypeptides are also referred to as “hybrid” polypeptides. The coding sequences includes those obtained from the same or from different species of organisms.

The term “fusion” when used in reference to a polypeptide refers to a chimeric protein containing a protein of interest joined to an exogenous protein fragment (the fusion partner). The fusion partner may serve various functions, including enhancement of solubility of the polypeptide of interest, as well as providing an “affinity tag” to allow purification of the recombinant fusion polypeptide from a host cell or from a supernatant or from both. If desired, the fusion partner may be removed from the protein of interest after or during purification.

The term “homolog” or “homologous” when used in reference to a polypeptide refers to a high degree of sequence identity between two polypeptides, or to a high degree of similarity between the three-dimensional structure or to a high degree of similarity between the active site and the mechanism of action. In a preferred embodiment, a homolog has a greater than 60% sequence identity, and more preferably greater than 75% sequence identity, and still more preferably greater than 90% sequence identity, with a reference sequence.

As applied to polypeptides, the term “substantial identity” means that two peptide sequences, when optimally aligned, such as by the programs GAP or BESTFIT using default gap weights, share at least 80 percent sequence identity, preferably at least 90 percent sequence identity, more preferably at least 95 percent sequence identity or more (e.g., 99 percent sequence identity). Preferably, residue positions which are not identical differ by conservative amino acid substitutions.

The terms “variant” and “mutant” when used in reference to a polypeptide refer to an amino acid sequence that differs by one or more amino acids from another, usually related polypeptide. The variant may have “conservative” changes, wherein a substituted amino acid has similar structural or chemical properties. One type of conservative amino acid substitutions refers to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, and asparagine-glutamine. More rarely, a variant may have “non-conservative” changes (e.g., replacement of a glycine with a tryptophan). Similar minor variations may also include amino acid deletions or insertions (in other words, additions), or both. Guidance in determining which and how many amino acid residues may be substituted, inserted or deleted without abolishing biological activity may be found using computer programs well known in the art, for example, DNAStar software. Variants can be tested in functional assays. Preferred variants have less than 10%, and preferably less than 5%, and still more preferably less than 2% changes (whether substitutions, deletions, and so on).

The term “gene” refers to a nucleic acid (e.g., DNA or RNA) sequence that comprises coding sequences necessary for the production of an RNA, or a polypeptide or its precursor (e.g., proinsulin). A functional polypeptide can be encoded by a full length coding sequence or by any portion of the coding sequence as long as the desired activity or functional properties (e.g., enzymatic activity, ligand binding, signal transduction, etc.) of the polypeptide are retained. The term “portion” when used in reference to a gene refers to fragments of that gene. The fragments may range in size from a few nucleotides to the entire gene sequence minus one nucleotide. Thus, “a nucleotide comprising at least a portion of a gene” may comprise fragments of the gene or the entire gene.

The term “gene” also encompasses the coding regions of a structural gene and includes sequences located adjacent to the coding region on both the 5′ and 3′ ends for a distance of about 1 kb on either end such that the gene corresponds to the length of the full-length mRNA. The sequences which are located 5′ of the coding region and which are present on the mRNA are referred to as 5′ non-translated sequences. The sequences which are located 3′ or downstream of the coding region and which are present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene which are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

In addition to containing introns, genomic forms of a gene may also include sequences located on both the 5′ and 3′ end of the sequences which are present on the RNA transcript. These sequences are referred to as “flanking” sequences or regions (these flanking sequences are located 5′ or 3′ to the non-translated sequences present on the mRNA transcript). The 5′ flanking region may contain regulatory sequences such as promoters and enhancers which control or influence the transcription of the gene. The 3′ flanking region may contain sequences which direct the termination of transcription, posttranscriptional cleavage and polyadenylation.

The term “heterologous gene” refers to a gene encoding a factor that is not in its natural environment (i.e., has been altered by the hand of man). For example, a heterologous gene includes a gene from one species introduced into another species. A heterologous gene also includes a gene native to an organism that has been altered in some way (e.g., mutated, added in multiple copies, linked to a non-native promoter or enhancer sequence, etc.). Heterologous genes may comprise a gene sequence that comprise cDNA forms of the gene; the cDNA sequences may be expressed in either a sense (to produce mRNA) or anti-sense orientation (to produce an anti-sense RNA transcript that is complementary to the mRNA transcript). Heterologous genes are distinguished from endogenous genes in that the heterologous gene sequences are typically joined to nucleotide sequences comprising regulatory elements such as promoters that are not found naturally associated with the gene for the protein encoded by the heterologous gene or with gene sequences in the chromosome, or are associated with portions of the chromosome not found in nature (e.g., genes expressed in loci where the gene is not normally expressed).

The term “polynucleotide” refers to a molecule comprised of two or more deoxyribonucleotides or ribonucleotides, preferably more than three, and usually more than ten. The exact size will depend on many factors, which in turn depends on the ultimate function or use of the polynucleotide. The polynucleotide may be generated in any manner, including chemical synthesis, DNA replication, reverse transcription, or a combination thereof. The term “oligonucleotide” generally refers to a short length of single-stranded polynucleotide chain usually less than 30 nucleotides long, although it may also be used interchangeably with the term “polynucleotide.”

The term “nucleic acid” refers to a polymer of nucleotides, or a polynucleotide, as described above. The term is used to designate a single molecule, or a collection of molecules. Nucleic acids may be single stranded or double stranded, and may include coding regions and regions of various control elements, as described below.

The term “region” or “portion” when used in reference to a nucleic acid molecule refers to a set of linked nucleotides which is less than the entire length of the molecule.

The term “links” when used in reference to a nucleic acid molecule refers to a region which joins two other regions or portions of the nucleic acid molecule. In an RNA molecule, such a linking region may join two other portions of the RNA molecule which are complementary to each other and which therefore can form a double stranded or duplex molecule in the regions of complementarity; such links are usually single stranded, and are referred to as “loops”.

The term “a polynucleotide having a nucleotide sequence encoding a gene” or “a polynucleotide having a nucleotide sequence encoding a gene “or ” a nucleic acid sequence encoding” a specified RNA molecule or polypeptide refers to a nucleic acid sequence comprising the coding region of a gene or in other words the nucleic acid sequence which encodes a gene product. The coding region may be present in either a cDNA, genomic DNA or RNA form. When present in a DNA form, the oligonucleotide, polynucleotide, or nucleic acid may be single-stranded (i.e., the sense strand) or double-stranded. Suitable control elements such as enhancers/promoters, splice junctions, polyadenylation signals, etc. may be placed in close proximity to the coding region of the gene if needed to permit proper initiation of transcription and/or correct processing of the primary RNA transcript. Alternatively, the coding region utilized in the expression vectors may contain endogenous enhancers/promoters, splice junctions, intervening sequences, polyadenylation signals, etc. or a combination of both endogenous and exogenous control elements.

The term “recombinant” when made in reference to a nucleic acid molecule refers to a nucleic acid molecule which is comprised of segments of nucleic acid joined together by means of molecular biological techniques. The term “recombinant” when made in reference to a protein or a polypeptide refers to a protein molecule which is expressed using a recombinant nucleic acid molecule.

The terms “complementary” and “complementarity” refer to polynucleotides (i.e., a sequence of nucleotides) related by the base-pairing rules. For example, for the sequence “A-G-T,” is complementary to the sequence “T-C-A.” Complementarity may be “partial,” in which only some of the nucleic acids′ bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods which depend upon binding between nucleic acids.

The term “homology” when used in relation to nucleic acids or amino acids refers to a degree of similarity or relatedness, as for example between base sequences in different nucleic acid sequences, or between base sequences in different regions of a nucleic acid sequence. There may be partial homology or complete homology (i.e., identity). “Sequence identity” refers to a measure of relatedness between two or more nucleic acids or proteins, and is given as a percentage with reference to the total comparison length. The identity calculation takes into account those nucleotide or amino acid residues that are identical and in the same relative positions in their respective larger sequences. Calculations of identity may be performed by algorithms contained within computer programs such as “GAP” (Genetics Computer Group, Madison, Wis.) and “ALIGN” (DNAStar, Madison, Wis.).

Experimental determination of homology of nucleic acids may be made by hybridization measurements. A partially complementary sequence is one that at least partially inhibits (or competes with) a completely complementary sequence from hybridizing to a target nucleic acid, and is referred to using the functional term “substantially homologous.” The inhibition of hybridization of the completely complementary sequence to the target sequence may be examined using a hybridization assay (Southern or Northern blot, solution hybridization and the like) under conditions of low stringency. A substantially homologous sequence or probe will compete for and inhibit the binding (i.e., the hybridization) of a sequence which is completely homologous to a target under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target which lacks even a partial degree of complementarity (e.g., less than about 30% identity); in the absence of non-specific binding, the probe will not hybridize to the second non-complementary target.

The following terms are used to describe the sequence relationships between two or more polynucleotides: “reference sequence”, “sequence identity”, “percentage of sequence identity”, and “substantial identity”. A “reference sequence” is a defined sequence used as a basis for a sequence comparison; a reference sequence may be a subset of a larger sequence, for example, as a segment of a full-length cDNA sequence given in a sequence listing or may comprise a complete gene sequence. Generally, a reference sequence is at least 20 nucleotides in length, frequently at least 25 nucleotides in length, and often at least 50 nucleotides in length. Since two polynucleotides may each (1) comprise a sequence (i.e., a portion of the complete polynucleotide sequence) that is similar between the two polynucleotides, and (2) may further comprise a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window”, as used herein, refers to a conceptual segment of at least 20 contiguous nucleotide positions wherein a polynucleotide sequence may be compared to a reference sequence of at least 20 contiguous nucleotides and wherein the portion of the polynucleotide sequence in the comparison window may comprise additions or deletions (i.e., gaps) of 20 percent or less as compared to the reference sequence (which does not comprise additions or deletions) for optimal alignment of the two sequences. Optimal alignment of sequences for aligning a comparison window may be conducted by the local homology algorithm of Smith and Waterman (Smith and Waterman, Adv. Appl. Math. 2: 482 (1981)) by the homology alignment algorithm of Needleman and Wunsch (Needleman and Wunsch, J. Mol. Biol. 48:443 (1970)), by the search for similarity method of Pearson and Lipman (Pearson and Lipman, Proc. Natl. Acad. Sci. (U.S.A.) 85:2444 (1988)), by computerized implementations of these algorithms (GAP, BESTFIT, FASTA, and TFASTA in the Wisconsin Genetics Software Package Release 7.0, Genetics Computer Group, 575 Science Dr., Madison, Wis.), or by inspection, and the best alignment (i.e., resulting in the highest percentage of homology over the comparison window) generated by the various methods is selected. The term “sequence identity” means that two polynucleotide sequences are identical (i.e., on a nucleotide-by-nucleotide basis) over the window of comparison. The term “percentage of sequence identity” is calculated by comparing two optimally aligned sequences over the window of comparison, determining the number of positions at which the identical nucleic acid base (e.g., A, T, C, G, U, or I) occurs in both sequences to yield the number of matched positions, dividing the number of matched positions by the total number of positions in the window of comparison (i.e., the window size), and multiplying the result by 100 to yield the percentage of sequence identity. The terms “substantial identity” as used herein denotes a characteristic of a polynucleotide sequence, wherein the polynucleotide comprises a sequence that has at least 85 percent sequence identity, preferably at least 90 to 95 percent sequence identity, more usually at least 99 percent sequence identity as compared to a reference sequence over a comparison window of at least 20 nucleotide positions, frequently over a window of at least 25-50 nucleotides, wherein the percentage of sequence identity is calculated by comparing the reference sequence to the polynucleotide sequence which may include deletions or additions which total 20 percent or less of the reference sequence over the window of comparison. The reference sequence may be a subset of a larger sequence, for example, as a segment of the full-length sequences of the compositions claimed in the present invention.

When used in reference to a double-stranded nucleic acid sequence such as a cDNA or genomic clone, the term “substantially homologous” refers to any probe that can hybridize to either or both strands of the double-stranded nucleic acid sequence under conditions of low to high stringency as described above.

When used in reference to a single-stranded nucleic acid sequence, the term “substantially homologous” refers to any probe that can hybridize (i.e., it is the complement of) the single-stranded nucleic acid sequence under conditions of low to high stringency as described above.

The term “hybridization” refers to the pairing of complementary nucleic acids. Hybridization and the strength of hybridization (i.e., the strength of the association between the nucleic acids) is impacted by such factors as the degree of complementary between the nucleic acids, stringency of the conditions involved, the Tₘ of the formed hybrid, and the G:C ratio within the nucleic acids. A single molecule that contains pairing of complementary nucleic acids within its structure is said to be “self-hybridized.”

The term “Tₘ” refers to the “melting temperature” of a nucleic acid. The melting temperature is the temperature at which a population of double-stranded nucleic acid molecules becomes half dissociated into single strands. The equation for calculating the Tₘ of nucleic acids is well known in the art. As indicated by standard references, a simple estimate of the Tₘ value may be calculated by the equation: Tₘ=81.5+0.41(% G+C), when a nucleic acid is in aqueous solution at 1 M NaCl (See e.g., Anderson and Young, Quantitative Filter Hybridization, in Nucleic Acid Hybridization (1985)). Other references include more sophisticated computations that take structural as well as sequence characteristics into account for the calculation of Tₘ.

As used herein the term “stringency” refers to the conditions of temperature, ionic strength, and the presence of other compounds such as organic solvents, under which nucleic acid hybridizations are conducted. With “high stringency” conditions, nucleic acid base pairing will occur only between nucleic acid fragments that have a high frequency of complementary base sequences. Thus, conditions of “low” stringency are often required with nucleic acids that are derived from organisms that are genetically diverse, as the frequency of complementary sequences is usually less.

“Low stringency conditions” when used in reference to nucleic acid hybridization comprise conditions equivalent to binding or hybridization at 42° C. in a solution consisting of 5×SSPE (43.8 g/l NaCl, 6.9 g/l NaH₂PO₄.H₂O and 1.85 g/l EDTA, pH adjusted to 7.4 with NaOH), 0.1% SDS, 5×Denhardt's reagent [50×Denhardt's contains per 500 ml: 5 g Ficoll (Type 400, Pharmacia), 5 g BSA (Fraction V; Sigma)) and 100 μg/ml denatured salmon sperm DNA followed by washing in a solution comprising 5×SSPE, 0.1% SDS at 42° C. when a probe of about 500 nucleotides in length is employed. “Medium stringency conditions” when used in reference to nucleic acid hybridization comprise conditions equivalent to binding or hybridization at 42° C. in a solution consisting of 5×SSPE (43.8 g/l NaCl, 6.9 g/l NaH₂PO₄.H₂O and 1.85 g/l EDTA, pH adjusted to 7.4 with NaOH), 0.5% SDS, 5×Denhardt's reagent and 100 μg/ml denatured salmon sperm DNA followed by washing in a solution comprising 1.0XSSPE, 1.0% SDS at 42° C. when a probe of about 500 nucleotides in length is employed.

“High stringency conditions” when used in reference to nucleic acid hybridization comprise conditions equivalent to binding or hybridization at 42° C. in a solution consisting of 5×SSPE (43.8 g/l NaCl, 6.9 g/l NaH₂PO₄.H₂O and 1.85 g/l EDTA, pH adjusted to 7.4 with NaOH), 0.5% SDS, 5×Denhardt's reagent and 100 pg/ml denatured salmon sperm DNA followed by washing in a solution comprising 0.1×SSPE, 1.0% SDS at 42° C. when a probe of about 500 nucleotides in length is employed.

It is well known that numerous equivalent conditions may be employed to comprise low stringency conditions; factors such as the length and nature (DNA, RNA, base composition) of the probe and nature of the target (DNA, RNA, base composition, present in solution or immobilized, etc.) and the concentration of the salts and other components (e.g., the presence or absence of formamide, dextran sulfate, polyethylene glycol) are considered and the hybridization solution may be varied to generate conditions of low stringency hybridization different from, but equivalent to, the above listed conditions. In addition, the art knows conditions that promote hybridization under conditions of high stringency (e.g., increasing the temperature of the hybridization and/or wash steps, the use of formamide in the hybridization solution, etc.).

“Amplification” is a special case of nucleic acid replication involving template specificity. It is to be contrasted with non-specific template replication (i.e., replication that is template-dependent but not dependent on a specific template). Template specificity is here distinguished from fidelity of replication (i.e., synthesis of the proper polynucleotide sequence) and nucleotide (ribo- or deoxyribo-) specificity. Template specificity is frequently described in terms of “target” specificity. Target sequences are “targets” in the sense that they are sought to be sorted out from other nucleic acid. Amplification techniques have been designed primarily for this sorting out.

Template specificity is achieved in most amplification techniques by the choice of enzyme. Amplification enzymes are enzymes that, under conditions they are used, will process only specific sequences of nucleic acid in a heterogeneous mixture of nucleic acid. For example, in the case of Q₁₃ replicase, MDV-1 RNA is the specific template for the replicase (Kacian et al., Proc. Natl. Acad. Sci. USA, 69:3038 (1972)). Other nucleic acid will not be replicated by this amplification enzyme. Similarly, in the case of T7 RNA polymerase, this amplification enzyme has a stringent specificity for its own promoters (Chamberlin et al., Nature, 228:227 (1970)). In the case of T4 DNA ligase, the enzyme will not ligate the two oligonucleotides or polynucleotides, where there is a mismatch between the oligonucleotide or polynucleotide substrate and the template at the ligation junction (Wu and Wallace, Genomics, 4:560 (1989)). Finally, Taq and Pfu polymerases, by virtue of their ability to function at high temperature, are found to display high specificity for the sequences bounded and thus defined by the primers; the high temperature results in thermodynamic conditions that favor primer hybridization with the target sequences and not hybridization with non-target sequences (H. A. Erlich (ed.), PCR Technology, Stockton Press (1989)).

The term “amplifiable nucleic acid” refers to nucleic acids that may be amplified by any amplification method. It is contemplated that “amplifiable nucleic acid” will usually comprise “sample template.”

The term “sample template” refers to nucleic acid originating from a sample that is analyzed for the presence of “target” (defined below). In contrast, “background template” is used in reference to nucleic acid other than sample template that may or may not be present in a sample. Background template is most often inadvertent. It may be the result of carryover, or it may be due to the presence of nucleic acid contaminants sought to be purified away from the sample. For example, nucleic acids from organisms other than those to be detected may be present as background in a test sample.

The term “primer” refers to an oligonucleotide, whether occurring naturally as in a purified restriction digest or produced synthetically, which is capable of acting as a point of initiation of synthesis when placed under conditions in which synthesis of a primer extension product which is complementary to a nucleic acid strand is induced, (i.e., in the presence of nucleotides and an inducing agent such as DNA polymerase and at a suitable temperature and pH). The primer is preferably single stranded for maximum efficiency in amplification, but may alternatively be double stranded. If double stranded, the primer is first treated to separate its strands before being used to prepare extension products. Preferably, the primer is an oligodeoxyribonucleotide. The primer is sufficiently long to prime the synthesis of extension products in the presence of the inducing agent. The exact lengths of the primers will depend on many factors, including temperature, source of primer and the use of the method.

The term “probe” refers to an oligonucleotide (i.e., a sequence of nucleotides), whether occurring naturally as in a purified restriction digest or produced synthetically, recombinantly or by PCR amplification, that is capable of hybridizing to another oligonucleotide of interest. A probe may be single-stranded or double-stranded. Probes are useful in the detection, identification and isolation of particular gene sequences. It is contemplated that any probe used in the present invention will be labeled with any “reporter molecule,” so that is detectable in any detection system, including, but not limited to enzyme (e.g., ELISA, as well as enzyme-based histochemical assays), fluorescent, radioactive, and luminescent systems. It is not intended that the present invention be limited to any particular detection system or label.

The term “target,” when used in reference to the polymerase chain reaction, refers to the region of nucleic acid bounded by the primers used for polymerase chain reaction. Thus, the “target” is sought to be sorted out from other nucleic acid sequences. A “segment” is defined as a region of nucleic acid within the target sequence.

The term “polymerase chain reaction” (“PCR”) refers to the method of K. B. Mullis U.S. Pat. Nos. 4,683,195, 4,683,202, and 4,965,188, that describe a method for increasing the concentration of a segment of a target sequence in a mixture of genomic DNA without cloning or purification. This process for amplifying the target sequence consists of introducing a large excess of two oligonucleotide primers to the DNA mixture containing the desired target sequence, followed by a precise sequence of thermal cycling in the presence of a DNA polymerase. The two primers are complementary to their respective strands of the double stranded target sequence. To effect amplification, the mixture is denatured and the primers then annealed to their complementary sequences within the target molecule. Following annealing, the primers are extended with a polymerase so as to form a new pair of complementary strands. The steps of denaturation, primer annealing, and polymerase extension can be repeated many times (i.e., denaturation, annealing and extension constitute one “cycle”; there can be numerous “cycles”) to obtain a high concentration of an amplified segment of the desired target sequence. The length of the amplified segment of the desired target sequence is determined by the relative positions of the primers with respect to each other, and therefore, this length is a controllable parameter. By virtue of the repeating aspect of the process, the method is referred to as the “polymerase chain reaction” (hereinafter “PCR”). Because the desired amplified segments of the target sequence become the predominant sequences (in terms of concentration) in the mixture, they are said to be “PCR amplified.”

With PCR, it is possible to amplify a single copy of a specific target sequence in genomic DNA to a level detectable by several different methodologies (e.g., hybridization with a labeled probe; incorporation of biotinylated primers followed by avidin-enzyme conjugate detection; incorporation of ³²P-labeled deoxynucleotide triphosphates, such as dCTP or dATP, into the amplified segment). In addition to genomic DNA, any oligonucleotide or polynucleotide sequence can be amplified with the appropriate set of primer molecules. In particular, the amplified segments created by the PCR process itself are, themselves, efficient templates for subsequent PCR amplifications.

The terms “PCR product,” “PCR fragment,” and “amplification product” refer to the resultant mixture of compounds after two or more cycles of the PCR steps of denaturation, annealing and extension are complete. These terms encompass the case where there has been amplification of one or more segments of one or more target sequences.

The term “amplification reagents” refers to those reagents (deoxyribonucleotide triphosphates, buffer, etc.), needed for amplification except for primers, nucleic acid template, and the amplification enzyme. Typically, amplification reagents along with other reaction components are placed and contained in a reaction vessel (test tube, microwell, etc.).

The term “reverse-transcriptase” or “RT-PCR” refers to a type of PCR where the starting material is mRNA. The starting mRNA is enzymatically converted to complementary DNA or “cDNA” using a reverse transcriptase enzyme. The cDNA is then used as a “template” for a “PCR” reaction

The term “gene expression” refers to the process of converting genetic information encoded in a gene into RNA (e.g., mRNA, rRNA, tRNA, or snRNA) through “transcription” of the gene (i.e., via the enzymatic action of an RNA polymerase), and, where the RNA encodes a protein, into protein, through “translation” of mRNA. Gene expression can be regulated at many stages in the process. “Up-regulation” or “activation” refers to regulation that increases the production of gene expression products (i.e., RNA or protein), while “down-regulation” or “repression” refers to regulation that decrease production. Molecules (e.g., transcription factors) that are involved in up-regulation or down-regulation are often called “activators” and “repressors,” respectively.

The terms “in operable combination”, “in operable order” and “operably linked” refer to the linkage of nucleic acid sequences in such a manner that a nucleic acid molecule capable of directing the transcription of a given gene and/or the synthesis of a desired protein molecule is produced. The term also refers to the linkage of amino acid sequences in such a manner so that a functional protein is produced.

The term “regulatory element” refers to a genetic element which controls some aspect of the expression of nucleic acid sequences. For example, a promoter is a regulatory element which facilitates the initiation of transcription of an operably linked coding region. Other regulatory elements are splicing signals, polyadenylation signals, termination signals, etc.

Transcriptional control signals in eukaryotes comprise “promoter” and “enhancer” elements. Promoters and enhancers consist of short arrays of DNA sequences that interact specifically with cellular proteins involved in transcription (Maniatis, et al., Science 236:1237, 1987). Promoter and enhancer elements have been isolated from a variety of eukaryotic sources including genes in yeast, insect, mammalian and plant cells. Promoter and enhancer elements have also been isolated from viruses and analogous control elements, such as promoters, are also found in prokaryotes. The selection of a particular promoter and enhancer depends on the cell type used to express the protein of interest. Some eukaryotic promoters and enhancers have a broad host range while others are functional in a limited subset of cell types (for review, see Voss, et al., Trends Biochem. Sci., 11:287, 1986; and Maniatis, et al., supra 1987).

The terms “promoter element,” “promoter,” or “promoter sequence” as used herein, refer to a DNA sequence that is located at the 5′ end (i.e. precedes) the protein coding region of a DNA polymer. The location of most promoters known in nature precedes the transcribed region. The promoter functions as a switch, activating the expression of a gene. If the gene is activated, it is said to be transcribed, or participating in transcription. Transcription involves the synthesis of mRNA from the gene. The promoter, therefore, serves as a transcriptional regulatory element and also provides a site for initiation of transcription of the gene into mRNA.

Promoters may be tissue specific or cell specific. The term “tissue specific” as it applies to a promoter refers to a promoter that is capable of directing selective expression of a nucleotide sequence of interest to a specific type of tissue (e.g., liver) in the relative absence of expression of the same nucleotide sequence of interest in a different type of tissue (e.g., brain). Tissue specificity of a promoter may be evaluated by, for example, operably linking a reporter gene to the promoter sequence to generate a reporter construct, introducing the reporter construct into the genome of an organism such that the reporter construct is integrated into every tissue of the resulting transgenic organism, and detecting the expression of the reporter gene (e.g., detecting mRNA, protein, or the activity of a protein encoded by the reporter gene) in different tissues of the transgenic organism. The detection of a greater level of expression of the reporter gene in one or more tissues relative to the level of expression of the reporter gene in other tissues shows that the promoter is specific for the tissues in which greater levels of expression are detected. The term “cell type specific” as applied to a promoter refers to a promoter which is capable of directing selective expression of a nucleotide sequence of interest in a specific type of cell in the relative absence of expression of the same nucleotide sequence of interest in a different type of cell within the same tissue. The term “cell type specific” when applied to a promoter also means a promoter capable of promoting selective expression of a nucleotide sequence of interest in a region within a single tissue. Cell type specificity of a promoter may be assessed using methods well known in the art, e.g., immunohistochemical staining. Briefly, tissue sections are embedded in paraffin, and paraffin sections are reacted with a primary antibody which is specific for the polypeptide or nucleotide product encoded by the nucleotide sequence of interest whose expression is controlled by the promoter. A labeled (e.g., peroxidase conjugated) secondary antibody which is specific for the primary antibody is allowed to bind to the sectioned tissue and specific binding detected (e.g., with avidin/biotin) by microscopy.

Promoters may be constitutive or regulatable. The term “constitutive” when made in reference to a promoter means that the promoter is capable of directing transcription of an operably linked nucleic acid sequence in the absence of a stimulus (e.g., heat shock, chemicals, light, etc.). Typically, constitutive promoters are capable of directing expression of a transgene in substantially any cell and any tissue. Exemplary promoters include but are not limited to pol III promoters, including promoters from either 7SL signal recognition particle RNA (srpRNA), 5S ribosomal RNA (rRNA), or U6 small nuclear RNA (snRNA) genes as described below, and tRNA, RNase P RNA, and adenovirus VA RNA pol III promoters as described in the following references, which are hereby incorporated in their entirety (Medina, M. F. C. and Joshi, S. (1999) Curr. Opin. Mol. Ther. 1: 580-594; Brummelkamp, T. R. et al. (2002) Science. 296: 550-553; McManus, M. T. et al. (2002). RNA. 8: 842-850). Promoters may be modified so as to possess different specificity.

In contrast, a “regulatable” or “inducible” promoter is one which is capable of directing a level of transcription of an operably linked nuclei acid sequence in the presence of a stimulus (e.g., heat shock, chemicals, light, etc.) which is different from the level of transcription of the operably linked nucleic acid sequence in the absence of the stimulus.

The enhancer and/or promoter may be “endogenous” or “exogenous” or “heterologous.” An “endogenous” enhancer or promoter is one that is naturally linked with a given gene in the genome. An “exogenous” or “heterologous” enhancer or promoter is one that is placed in juxtaposition to a gene by means of genetic manipulation (i.e., molecular biological techniques) such that transcription of the gene is directed by the linked enhancer or promoter. For example, an endogenous promoter in operable combination with a first gene can be isolated, removed, and placed in operable combination with a second gene, thereby making it a “heterologous promoter” in operable combination with the second gene. A variety of such combinations are contemplated (e.g., the first and second genes can be from the same species, or from different species).

The presence of “splicing signals” on an expression vector often results in higher levels of expression of the recombinant transcript in eukaryotic host cells. Splicing signals mediate the removal of introns from the primary RNA transcript and consist of a splice donor and acceptor site (Sambrook, et al., Molecular Cloning: A Laboratory Manual, 2nd ed., Cold Spring Harbor Laboratory Press, New York (1989) pp. 16.7-16.8). A commonly used splice donor and acceptor site is the splice junction from the 16S RNA of SV40.

Efficient expression of recombinant DNA sequences in eukaryotic cells generally requires expression of signals directing the efficient termination and polyadenylation of the resulting transcript. Transcription termination signals are generally found downstream of the polyadenylation signal and are a few hundred nucleotides in length. The term “poly(A) site” or “poly(A) sequence” as used herein denotes a DNA sequence which directs both the termination and polyadenylation of the nascent RNA transcript. Efficient polyadenylation of the recombinant transcript is desirable, as transcripts lacking a poly(A) tail are unstable and are rapidly degraded. The poly(A) signal utilized in an expression vector may be “heterologous” or “endogenous.” An endogenous poly(A) signal is one that is found naturally at the 3′ end of the coding region of a given gene in the genome. A heterologous poly(A) signal is one which has been isolated from one gene and positioned 3′ to another gene. A commonly used heterologous poly(A) signal is the SV40 poly(A) signal. The SV40 poly(A) signal is contained on a 237 bp BamHIBclI restriction fragment and directs both termination and polyadenylation (Sambrook, supra, at 16.6-16.7).

The term “vector” refers to nucleic acid molecules that transfer DNA segment(s) from one cell to another. The term “vehicle” is sometimes used interchangeably with “vector.” A vector may be used to transfer an expression cassette into a cell; in addition or alternatively, a vector may comprise additional genes, including but not limited to genes which encode marker proteins, by which cell transfection can be determined, selection proteins, be means of which transfected cells may be selected from non- transfected cells, or reporter proteins, by means of which an effect on expression or activity or function of the reporter protein can be monitored.

The term “expression cassette” refers to a recombinant DNA molecule containing a desired coding sequence and appropriate nucleic acid sequences necessary for the expression of the operably linked coding sequence in a particular host organism. Nucleic acid sequences necessary for expression in prokaryotes usually include a promoter, an operator (optional), and a ribosome binding site, often along with other sequences. Eukaryotic cells are known to utilize promoters, enhancers, and termination and polyadenylation signals

The term “expression vector” refers to a vector comprising one or more expression cassettes. Such expression cassettes include those of the present invention, where expression results in an siRNA transcript.

The term “transfection” refers to the introduction of foreign DNA into cells. Transfection may be accomplished by a variety of means known to the art including calcium phosphate-DNA co-precipitation, DEAE-dextran-mediated transfection, polybrene-mediated transfection, glass beads, electroporation, microinjection, liposome fusion, lipofection, protoplast fusion, viral infection, biolistics (i.e., particle bombardment) and the like.

The term “stable transfection” or “stably transfected” refers to the introduction and integration of foreign DNA into the genome of the transfected cell. The term “stable transfectant” refers to a cell that has stably integrated foreign DNA into the genomic DNA.

The term “transient transfection” or “transiently transfected” refers to the introduction of foreign DNA into a cell where the foreign DNA fails to integrate into the genome of the transfected cell. The foreign DNA persists in the nucleus of the transfected cell for several days. During this time the foreign DNA is subject to the regulatory controls that govern the expression of endogenous genes in the chromosomes. The term “transient transfectant” refers to cells that have taken up foreign DNA but have failed to integrate this DNA.

The term “calcium phosphate co-precipitation” refers to a technique for the introduction of nucleic acids into a cell. The uptake of nucleic acids by cells is enhanced when the nucleic acid is presented as a calcium phosphate-nucleic acid co-precipitate. The original technique of Graham and van der Eb (Graham and van der Eb, Virol., 52:456 (1973)), has been modified by several groups to optimize conditions for

The terms “infecting” and “infection” when used with a bacterium refer to co-incubation of a target biological sample, (e.g., cell, tissue, etc.) with the bacterium under conditions such that nucleic acid sequences contained within the bacterium are introduced into one or more cells of the target biological sample.

The terms “bombarding, “bombardment,” and “biolistic bombardment” refer to the process of accelerating particles towards a target biological sample (e.g., cell, tissue, etc.) to effect wounding of the cell membrane of a cell in the target biological sample and/or entry of the particles into the target biological sample. Methods for biolistic bombardment are known in the art (e.g., U.S. Pat. No. 5,584,807, the contents of which are incorporated herein by reference), and are commercially available (e.g., the helium gas-driven microprojectile accelerator (PDS-1000/He, BioRad).

The term “transgene” as used herein refers to a foreign gene that is transferred or placed into an organism, as for example by introducing the foreign gene into newly fertilized eggs or early embryos. The term “foreign gene” refers to any nucleic acid (e.g., gene sequence) that is introduced into the genome of an animal by experimental manipulations and may include gene sequences found in that animal so long as the introduced gene does not reside in the same location as does the naturally-occurring gene. The term “transgene” and “foreign gene” may be used interchangeably.

The term “host cell” refers to any cell capable of replicating and/or transcribing and/or translating a heterologous gene. Thus, a “host cell” refers to any eukaryotic or prokaryotic cell (e.g., bacterial cells such as E. coli, yeast cells, mammalian cells, avian cells, amphibian cells, plant cells, fish cells, and insect cells), whether located in vitro or in vivo. For example, host cells may be located in a transgenic animal.

The terms “transformants” or “transformed cells” include the primary transformed cell and cultures derived from that cell without regard to the number of transfers. All progeny may not be precisely identical in DNA content, due to deliberate or inadvertent mutations. Mutant progeny that have the same functionality as screened for in the originally transformed cell are included in the definition of transformants.

The term “selectable marker” refers to a gene which encodes an enzyme having an activity that confers resistance to an antibiotic or drug upon the cell in which the selectable marker is expressed, or which confers expression of a trait which can be detected (e.g.., luminescence or fluorescence). Selectable markers may be “positive” or “negative.” Examples of positive selectable markers include the neomycin phosphotrasferase (NPTII) gene which confers resistance to G418 and to kanamycin, and the bacterial hygromycin phosphotransferase gene (hyg), which confers resistance to the antibiotic hygromycin. Negative selectable markers encode an enzymatic activity whose expression is cytotoxic to the cell when grown in an appropriate selective medium. For example, the HSV-tk gene is commonly used as a negative selectable marker. Expression of the HSV-tk gene in cells grown in the presence of gancyclovir or acyclovir is cytotoxic; thus, growth of cells in selective medium containing gancyclovir or acyclovir selects against cells capable of expressing a functional HSV TK enzyme.

The term “reporter gene” refers to a gene encoding a protein that may be assayed. Examples of reporter genes include, but are not limited to, luciferase (See, e.g., deWet et al., Mol. Cell. Biol. 7:725 (1987) and U.S. Pat. Nos.,6,074,859; 5,976,796; 5,674,713; and 5,618,682; all of which are incorporated herein by reference), green fluorescent protein (e.g., GenBank Accession Number U43284; a number of GFP variants are commercially available from ClonTech Laboratories, Palo Alto, Calif.), chloramphenicol acetyltransferase, β-galactosidase, alkaline phosphatase, and horse radish peroxidase.

The term “wild-type” when made in reference to a gene refers to a gene which has the characteristics of a gene isolated from a naturally occurring source. The term “wild- type” when made in reference to a gene product refers to a gene product which has the characteristics of a gene product isolated from a naturally occurring source. The term “naturally-occurring” as used herein as applied to an object refers to the fact that an object can be found in nature. For example, a polypeptide or polynucleotide sequence that is present in an organism (including viruses) that can be isolated from a source in nature and which has not been intentionally modified by man in the laboratory is naturally-occurring. A wild-type gene is that which is most frequently observed in a population and is thus arbitrarily designated the “normal” or “wild-type” form of the gene. In contrast, the term “modified” or “mutant” when made in reference to a gene or to a gene product refers, respectively, to a gene or to a gene product which displays modifications in sequence and/or functional properties (i.e., altered characteristics) when compared to the wild-type gene or gene product. It is noted that naturally-occurring mutants can be isolated; these are identified by the fact that they have altered characteristics when compared to the wild-type gene or gene product.

The term “antisense” refers to a deoxyribonucleotide sequence whose sequence of deoxyribonucleotide residues is in reverse 5′ to 3′ orientation in relation to the sequence of deoxyribonucleotide residues in a sense strand of a DNA duplex. A “sense strand” of a DNA duplex refers to a strand in a DNA duplex which is transcribed by a cell in its natural state into a “sense mRNA.” Thus an “antisense” sequence is a sequence having the same sequence as the non-coding strand in a DNA duplex. The term “antisense RNA” refers to a RNA transcript that is complementary to all or part of a target primary transcript or mRNA; many antisense RNAs block the expression of a target gene by interfering with the processing, transport and/or translation of its primary transcript, for example mRNA. The complementarity of an antisense RNA may be with any part of the specific gene transcript, i.e., at the 5′ non-coding sequence, 3′ non-coding sequence, introns, or the coding sequence. In addition, antisense RNA may contain regions of ribozyme sequences that increase the efficacy of antisense RNA to block gene expression. “Ribozyme” refers to a catalytic RNA and includes sequence-specific endoribonucleases. “Antisense inhibition” refers to the production of antisense RNA transcripts capable of preventing the expression of the target protein, or of preventing the function of a target RNA.

The term “siRNAs” refers to short interfering RNAs. In some embodiments, siRNAs comprise a duplex, or double-stranded region, where each strand of the double stranded region is about 18 to about 25 nucleotides long; the double stranded region can be as short as 16, and as long as 29, base pairs long, where the length is determined by the antisense strand. Often siRNAs contain from about two to four unpaired nucleotides at the 3′ end of each strand. siRNAs appear to function as key intermediaries in triggering RNA interference in invertebrates and in vertebrates, and in triggering sequence-specific RNA degradation during posttranscriptional gene silencing in plants. At least one strand of the duplex or double-stranded region of a siRNA is substantially homologous to or substantially complementary to a target RNA molecule. The strand complementary to a target RNA molecule is the “antisense strand;” the strand homologous to the target RNA molecule is the “sense strand,” and is also complementary to the siRNA antisense strand. One strand of the double stranded region need not be the exact length of the opposite strand; thus, one strand may have at least one fewer nucleotides than the opposite complementary strand, resulting in a “bubble” or at least one unmatched base in the opposite strand. One strand of the double stranded region need not be exactly complementary to the opposite strand; thus, the strand, preferably the sense strand, may have at least one mismatched base-pair.

siRNAs may also contain additional sequences; non-limiting examples of such sequences include linking sequences, or loops, which connect the two strands of the duplex region. This form of siRNAs may be referred to “si-like RNA,” “short hairpin siRNA,” where the short refers to the duplex region of the siRNA, or “hairpin siRNA.” Additional non-limiting examples of additional sequences present in siRNAs include stem and other folded structures. The additional sequences may or may not have known functions; non-limiting examples of such functions include increasing stability of an siRNA molecule, or providing a cellular destination signal.

The term “target RNA molecule” refers to an RNA molecule to which at least one strand of the short double-stranded region of an siRNA is complementary. Typically, when such complementarity is about 100%, the siRNA is able to silence or inhibit expression of the target RNA molecule. Although it is believed that processed mRNA is a target of siRNA, the present invention is not limited to any particular hypothesis, and such hypotheses are not necessary to practice the present invention. Thus, it is contemplated that other RNA molecules may also be targets of siRNA. Such targets include unprocessed mRNA, ribosomal RNA, and viral RNA genomes.

The term “ds siRNA” refers to a siRNA molecule which comprises two separate unlinked strands of RNA which form a duplex structure, such that the siRNA molecule comprises two RNA polynucleotides.

The term “hairpin siRNA” refers to a siRNA molecule which comprises at least one duplex region where the strands of the duplex are connected or contiguous at one or both ends, such that the siRNA molecule comprises a single RNA polynucleotide. The antisense sequence, or sequence which is complementary to a target RNA, comprises at least a part of the at least one double stranded region.

The term “cellular destination signal” or “destination signal” is a portion of an RNA molecule that directs the transport of an RNA molecule out of the nucleus, or that directs the retention of an RNA molecule in the nucleus; such signals may also direct an RNA molecule to a particular subcellular location. Such a signal may be an encoded signal, or it might be added post-transciptionally. Alternatively a cellular destination signal may be determined by the nature of the transcriptional promoter from which an RNA molecule is transcribed; in some examples, unless a specific subcellular destination signal (such as a cytoplasmic or nucleoplasmic) is embedded in an RNA transcript, the RNA product transcribed under control of the promoter is retained or stays in the nucleus, or is nucleoplasmicly destined. A destination signal also includes the absence of a signal, such that the default location of the transcriptional product is the location of the transcription; in other words, the transcribed RNA product generally remains in the subcellular location in which it is transcribed. Exemplary destination signals include those from the 7SL cassette or a known nucleolar snRNA, which direct transcribed siRNA product to the cytoplasm or the nucleolus, respectively, and that in the nature of the U6 promoter, which directs the retention of the siRNA product in the nucleus.

The term “enhancing the function” when used in reference to an siRNA molecule means that the effectiveness of an siRNA molecule in silencing gene expression is increased. Such enhancements include but are not limited to increased rates of formation of an siRNA molecule, decreased susceptibility to degradation, and increased transport throughout the cell. An increased rate of formation might result from a transcript which possesses sequences which enhance folding or the formation of a duplex strand.

The term “RNA interference” or “RNAi” refers to the silencing or decreasing expression, or inhibition of expression, of gene expression by siRNAs. It is the process of sequence-specific, post-transcriptional gene silencing in animals and plants, initiated by siRNA that is homologous in its duplex region to the sequence of the silenced gene or that is complementary in its duplex region to the transcriptional product of the silenced gene. The gene may be endogenous or exogenous to the organism, present integrated into a chromosome or present in a transfection vector which is not integrated into the genome. The expression of the silenced gene is either completely or partially inhibited.

The term “posttranscriptional gene silencing” or “PTGS” refers to silencing of gene expression in plants after transcription, and appears to involve the specific degradation of mRNAs synthesized from gene repeats.

The term “sequence-nonspecific gene silencing” refers to silencing gene expression in mammalian cells after transcription, and is induced by dsRNA of greater than about 30 base pairs. This appears to be due to an interferon response, in which dsRNA of greater than about 30 base pairs binds and activates the protein PKR and 2′,5′-oligonucleotide synthetase (2′,5′-AS). Activated PKR stalls translation by phosphorylation of the translation initiation factors eIF2alpha, and activated 2′,5′-AS causes mRNA degradation by 2′,5′-oligonucleeotide-activated ribonuclease L. These responses are intrinsically sequence-nonspecific to the inducing dsRNA.

The term “overexpression” refers to the production of a gene product in transgenic organisms that exceeds levels of production in normal or non-transformed organisms. The term “cosuppression” refers to the expression of a foreign gene which has substantial homology to an endogenous gene resulting in the suppression of expression of both the foreign and the endogenous gene. As used herein, the term “altered levels” refers to the production of gene product(s) in transgenic organisms in amounts or proportions that differ from that of normal or non-transformed organisms.

The terms “overexpression” and “overexpressing” and grammatical equivalents, are used in reference to levels of mRNA to indicate a level of expression approximately 3-fold higher than that typically observed in a given tissue in a control or non-transgenic animal. Levels of mRNA are measured using any of a number of techniques known to those skilled in the art including, but not limited to Northern blot analysis (See, Example 10, for a protocol for performing Northern blot analysis). Appropriate controls are included on the Northern blot to control for differences in the amount of RNA loaded from each tissue analyzed (e.g., the amount of 28S rRNA, an abundant RNA transcript present at essentially the same amount in all tissues, present in each sample can be used as a means of normalizing or standardizing the RAD50 mRNA-specific signal observed on Northern blots).

The terms “Southern blot analysis” and “Southern blot” and “Southern” refer to the analysis of DNA on agarose or acrylamide gels in which DNA is separated or fragmented according to size followed by transfer of the DNA from the gel to a solid support, such as nitrocellulose or a nylon membrane. The immobilized DNA is then exposed to a labeled probe to detect DNA species complementary to the probe used. The DNA may be cleaved with restriction enzymes prior to electrophoresis. Following electrophoresis, the DNA may be partially depurinated and denatured prior to or during transfer to the solid support. Southern blots are a standard tool of molecular biologists (J. Sambrook et al. (1989) Molecular Cloning: A Laboratory Manual, Cold Spring Harbor Press, N.Y., pp 9.31-9.58).

The term “Northern blot analysis” and “Northern blot” and “Northern” as used herein refer to the analysis of RNA by electrophoresis of RNA on agarose gels to fractionate the RNA according to size followed by transfer of the RNA from the gel to a solid support, such as nitrocellulose or a nylon membrane. The immobilized RNA is then probed with a labeled probe to detect RNA species complementary to the probe used. Northern blots are a standard tool of molecular biologists (J. Sambrook, et al. (1989) supra, pp 7.39-7.52).

The terms “Western blot analysis” and “Western blot” and “Western” refers to the analysis of protein(s) (or polypeptides) immobilized onto a support such as nitrocellulose or a membrane. A mixture comprising at least one protein is first separated on an acrylamide gel, and the separated proteins are then transferred from the gel to a solid support, such as nitrocellulose or a nylon membrane. The immobilized proteins are exposed to at least one antibody with reactivity against at least one antigen of interest. The bound antibodies may be detected by various methods, including the use of radiolabeled antibodies.

The term “antigenic determinant” as used herein refers to that portion of an antigen that makes contact with a particular antibody (i.e., an epitope). When a protein or fragment of a protein is used to immunize a host animal, numerous regions of the protein may induce the production of antibodies that bind specifically to a given region or three-dimensional structure on the protein; these regions or structures are referred to as antigenic determinants. An antigenic determinant may compete with the intact antigen (i.e., the “immunogen” used to elicit the immune response) for binding to an antibody.

The term “isolated” when used in relation to a nucleic acid, as in “an isolated oligonucleotide” refers to a nucleic acid sequence that is identified and separated from at least one contaminant nucleic acid with which it is ordinarily associated in its natural source. Isolated nucleic acid is present in a form or setting that is different from that in which it is found in nature. In contrast, non-isolated nucleic acids, such as DNA and RNA, are found in the state they exist in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNA s which encode a multitude of proteins. However, isolated nucleic acid encoding a particular protein includes, by way of example, such nucleic acid in cells ordinarily expressing the protein, where the nucleic acid is in a chromosomal location different from that of natural cells, or is otherwise flanked by a different nucleic acid sequence than that found in nature. The isolated nucleic acid or oligonucleotide may be present in single-stranded or double-stranded form. When an isolated nucleic acid or oligonucleotide is to be utilized to express a protein, the oligonucleotide will contain at a minimum the sense or coding strand (i.e., the oligonucleotide may single-stranded), but may contain both the sense and anti-sense strands (i.e., the oligonucleotide maybe double-stranded).

The term “purified” refers to molecules, either nucleic or amino acid sequences, that are removed from their natural environment, isolated or separated. An “isolated nucleic acid sequence” is therefore a purified nucleic acid sequence. “Substantially purified” molecules are at least 60% free, preferably at least 75% free, and more preferably at least 90% free from other components with which they are naturally associated. As used herein, the term “purified” or “to purify” also refers to the removal of contaminants from a sample. The removal of contaminating proteins results in an increase in the percent of polypeptide of interest in the sample. In another example, recombinant polypeptides are expressed in plant, bacterial, yeast, or mammalian host cells and the polypeptides are purified by the removal of host cell proteins; the percent of recombinant polypeptides is thereby increased in the sample.

The term “sample” is used in its broadest sense. In one sense it can refer to a plant cell or tissue. In another sense, it is meant to include a specimen or culture obtained from any source, as well as biological and environmental samples. Biological samples may be obtained from plants or animals (including humans) and encompass fluids, solids, tissues, and gases. Environmental samples include environmental material such as surface matter, soil, water, and industrial samples. These examples are not to be construed as limiting the sample types applicable to the present invention. The term “sample” is used in its broadest sense. In one sense it can refer to a biopolymeric material. In another sense, it is meant to include a specimen or culture obtained from any source, as well as biological and environmental samples. Biological samples may be obtained from animals (including humans) and encompass fluids, solids, tissues, and gases. Biological samples include blood products, such as plasma, serum and the like. Environmental samples include environmental material such as surface matter, soil, water, crystals and industrial samples. These examples are not to be construed as limiting the sample types applicable to the present invention.

### Detailed Description of the Invention

The present invention provides compositions and methods for intracellular expression and delivery of siRNAs in mammalian cells. In some aspects of the present inventions, the compositions comprise an expression cassette comprising a promoter and a gene which encodes an siRNA which upon transcription forms a short RNA containing a double stranded RNA (dsRNA) of about 18 to about 25 base pairs long. The double stranded siRNA may further comprise a loop of single stranded RNA, where the loop joins each strand at one end of the double strand; the loop may be as small as about 3 to 4 nucleotides long. In other embodiments of the present invention, the loop comprises additional nucleotide sequences which may contain stretches of double stranded RNA as well as stretches of single stranded RNA; in particular embodiments, the loop comprises a tRNA. The loop may be subject to processing in vivo, such as cleavage. In other embodiments, either the promoter or the siRNA or both may comprise a cellular destination signal. The target of the siRNA may be an endogenous gene, an exogenous gene, such as a viral or pathogenic gene or a transfected gene, or a gene of unknown function. In other aspects of the present invention, the compositions comprise at least two expression cassettes, each of which expresses one strand of siRNA which, when combined in the cell with the other strand expressed from the other cassette, forms a complete double stranded RNA; such RNA is characterized as described above.

In other aspects of the present invention, the compositions comprise a double stranded siRNA which further comprises a loop of single stranded RNA, where the loop joins each strand at one end of the double strand; the loop may be as small as about 3 to 4 nucleotides long. In other embodiments of the present invention, the loop comprises additional nucleotide sequences which may contain stretches of double stranded RNA as well as stretches of single stranded RNA; in some embodiments, the loop comprises a tRNA. The loop may be subject to processing in vivo, such as cleavage. In other embodiments, either the promoter or the siRNA or both may comprise a cellular destination signal. The target of the siRNA may be an endogenous gene, an exogenous gene, such as a viral or pathogenic gene or a transfected gene, or a gene of unknown function.

In yet other aspects of the present invention, the compositions comprise a vector comprising at least one expression cassette as described above; the vectors may further comprise marker genes, reporter genes, selection genes, or genes of interest, such as experimental genes. In yet other aspects of the present invention, the compositions comprise cells transfected by the expression cassettes of the present invention, or by a vector of the present invention. The cells are transfected transiently or stably. The cells are cultured mammalian cells, preferably human cells, or they are tissue, organ, or organismal cells. In yet further aspects of the present invention, the composition comprises a double stranded siRNA of about 18 to about 25 base pairs long, where the double strand is joined at one end by a loop of single stranded RNA; the loop may be as small as about 3 to 4 nucleotides long. In other embodiments of the present invention, the loop comprises additional nucleotide sequences which may contain stretches of double stranded RNA as well as stretches of single stranded RNA; in some embodiments, the loop comprises a tRNA. The loop may be subject to processing in vivo, such as cleavage.

The present invention also provides methods of transfecting a mammalian cell with an expression cassette or with a vector as described above. The present invention also provides methods of expressing siRNA in a mammalian cell by transfecting the cell with an expression cassette or with a vector as described above. The present invention also provides methods of silencing a gene in a mammalian cell by transfecting the cell with an expression cassette or with a vector as described above, where the siRNA encoded by the expression cassette targets a gene in the mammalian cell. In these methods, the cell is transfected transiently or stably, and the cell is a cultured mammalian cell, preferably a human cell, or it is a tissue, organ, or organismal cell. Moreover, in these methods, the target of the siRNA may be an endogenous gene, an exogenous gene, such as a viral or pathogenic gene or a transfected gene, or a gene of unknown function. Furthermore, in the methods the siRNA encoded by the expression cassette or vector upon transcription forms a double stranded RNA of about 18 to about 25 base pairs long. The double stranded RNA may further comprise a loop of single stranded RNA, where the loop joins the double strand at one end; the loop may be as small as about 3 to 4 nucleotides long. In other embodiments of the present invention, the loop comprises additional nucleotide sequences which may contain stretches of double stranded RNA as well as stretches of single stranded RNA; in some embodiments, the loop comprises a tRNA. The loop may be subject to processing in vivo, such as cleavage.

The following description first describes the development of the present invention. This is then followed by separate descriptions of the compositions and methods of the present invention, and applications of the present invention.

I. Development of the Invention

During development of the present invention, several methods of expressing siRNAs in mammalian cells were initially tested. These methods involved considerations of intracellular localization, RNA transcript size and structure, target sequences, and selection of effective promoters. Neither the expression nor the localization of siRNA in vivo has been reported; both expression and localization involved considerable unpredictability.

To successfully deliver effective RNA inhibitors to their intended targets in the cell, it is necessary to first identify factors which result in transcription of effective siRNAs. These factors include promoter strategies, structural strategies, localization strategies, and target sequence strategies. Because the field of siRNA is very new, little is known about the mechanism of how siRNA works, and where within the cell it occurs. Work on C. elegans and Drosophila indicates that RNA interference operates in the cytoplasm, supporting the idea that the target is processed mRNA (Carthew, R W (2001) Curr Opin Cell Biol 13(2): 244-248). On the other hand, very indirect and circumstantial evidence from plant postranscriptional gene silencing (PTGS) suggests that at least some aspects of RNAi may be localized to the nucleus. In plants, PTSG appears to be a viral suppressor and thus a mechanism of protection from viral infection; this hypothesis is supported by the evolutions of viral suppressors of PTSG (and host counter-measures against the viral suppressors). As an example, the HC-Pro protein of potato virus Y blocks maintenance of PTGS, whereas the 2b protein, a PTGS suppressor from cucumber mosaic virus, prevents its initiation. The 2b protein is localized to the nucleus, suggesting to one author that at least the early steps in silencing, perhaps the generation of dsRNA from a transgene or its processing into an active interfering molecule, may be nuclear (Zamore, P C (2001) Nat Struct Biol 8(9): 746-750). However, this does not exclude a cytoplasmic site of action of siRNA, which interpretation is supported by most of the data. Moreover, the mechanism of the siRNA mediated interference process in mammalian cells is described as remaining to be uncovered, as silencing might occur either post-transcriptionally and/or transcriptionally (Elbashir et al. (2001) Nature 411: 494-498).

Thus, one aspect of the effectiveness of siRNAs is the design of a gene which results in an RNA transcript with the appropriate subcellular location, as the correct cellular location of the RNA may be critical. For example, promoters for pol III are often found within the coding region of the gene, and in these cases, require the inclusion of extensive sequences from the pol III gene in the RNA transcripts. In addition, endogenous sequences from the normal transcript can provide enhanced stability of the RNA and direct the RNA to particular subcellular locations. These localization elements are often composed of relatively small regions of RNA that are recognized by cellular proteins, that in turn have localization signals and provide the vehicles by which the bound RNAs are transported. The inclusion of these domains of the RNA in the product of an expression cassette allows a potentially therapeutic RNA to be ‘carried’ to various locales within the cell. Natural pol III products are found in the cytoplasm, nucleoplasm, and nucleolus, allowing all three different subcellular compartments to be targeted using po III-based expression systems. This is especially useful because it is not always possible to predict the subcellular location in which a particular target will be most accessible.

Ribozymes provide an example of the importance of cellular localization; although they have been studied extensively in vitro, ribozymes are frequently not effective in cells (Rossi, J. J. (1998) BioDrugs. 9: 1-10). One of the reasons proposed for their lack of effectiveness is that they are not in the same subcellular location as their substrate. In cases where localization has been determined, a correlation between effective RNA locations and expected target locations has been observed (Medina, M. F. C. and Joshi, S. (1999) Curr. Opin. Mol. Ther. 1: 580-594; and Paul, C. P. et al. (1999) In Intracellular Ribozyme Applications: Principles and Protocols (J. J. Rossi and L. A. Couture, Eds.), pp. 93-102). Thus, it is contemplated that the success of an RNA designed to perform a particular function within the cell depends upon its ability to reach its target in addition to its capacity to recognize the target and perform the intended function.

Several different cassettes were designed for expression and delivery of siRNA to different cellular locations. These expression cassettes contained promoters from either 7SL signal recognition particle RNA (srpRNA), 5S ribosomal RNA (rRNA), or U6 small nuclear RNA (snRNA) genes, as well as additional gene sequences of varying lengths. The sequences contained what are believed to be destination signals for the transcribed RNA molecules, as well as structures which are suspected to confer increased stability upon the resulting RNA transcript. Generally, unless the transcript is deliberately translocated out of the nucleus, the default location of RNA transcripts appears to be the nucleus, as described below. However, the presence of certain signals in RNA transcripts is contemplated to result in the transport of these RNA transcripts out of the nucleus.

Expression cassettes which include the promoter and additional gene sequence of the U6 snRNA gene have been reported for expression of small RNA inserts, which included ribozymes, simple antisense clones, and aptamers (Good, P D et al. (1997) Gene Therapy 4: 45-54; and Bertrand, E et al. (1997) RNA 3: 75-88). U6 is a small, stable RNA that exists as an abundant nuclear ribonucleoprotein (U6 snRNP) in all human cells, where it plays central roles in both spliceosome assembly and catalysis in nuclear pre-mRNA splicing. All of the major transcriptional promoter elements for RNA polymerase III (pol III) are upstream of the transcription start. (Danzeiser D A et al. (1993) Molecular Cell Biol 13(8):4670-4678; Kunkel G R et al. (1986) Proc Natl Acad Sci USA 83:8575-8579; Kunkel GR, and Pederson T. (1989) Nucleic Acids Res 17(18):7371-7379; Noonberg SB et al. (1994) Nucleic Acids Res 22(14):2830-2836). This upstream U6 promoter strategy confers a potential advantage over the use of other pol III promoters (such as 5S rRNA and tRNA-classes), where the promoters are intragenic, and therefore any insert RNA is expressed as a fusion with the RNA sequences encoding the intragenic promoters. An additional advantage of the U6 gene is that individual genes are heavily expressed in human cells. Transcription of only a few copies of the U6 gene (out of 200 or more pseudogenes, Hayashi K. (1981) Nucleic Acids Res 9(14)3379-3389) results in roughly 400,000 copies of RNA per cell (Weinberg R A, and Penman S. (1968) J Mol Biol 38:289-304). The U6 primary transcript is normally trapped in the nucleus, appearing both in the nucleoplasm and in localized nuclear “speckles” (Carmo-Fonseca M, et al. (1992) J Cell Biol 117(1): 1-14). This is in contrast to other small nuclear RNAs synthesized by pol II, which exit to the cytoplasm and need to acquire an extensive protein complex bound to internal RNA sequences for re-entry into the nucleus (Andersen J, and Zieve G W. (1991) BioEssays 13(2):57-64; Hamm J, et al. (1990) Cell 62(3):569-577).

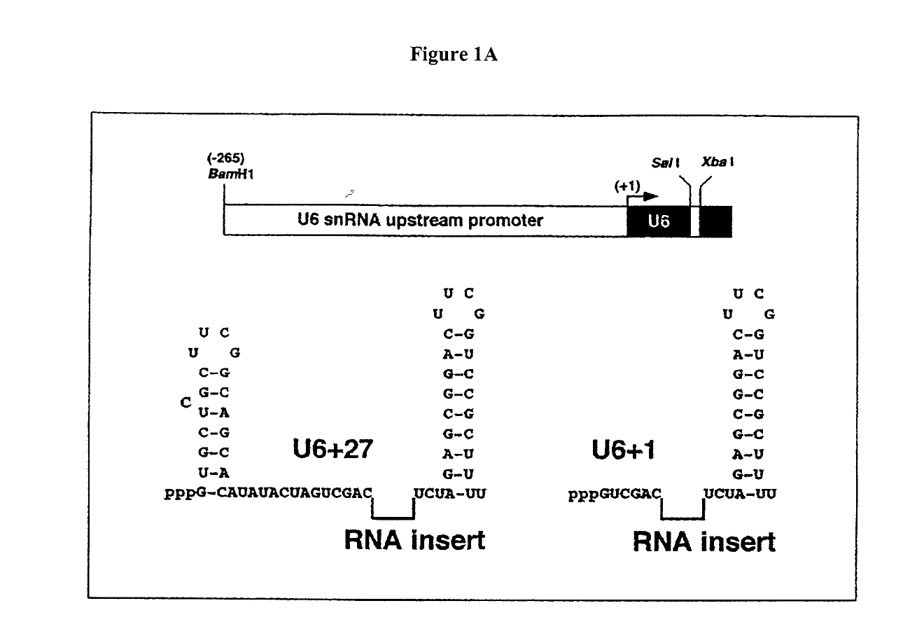

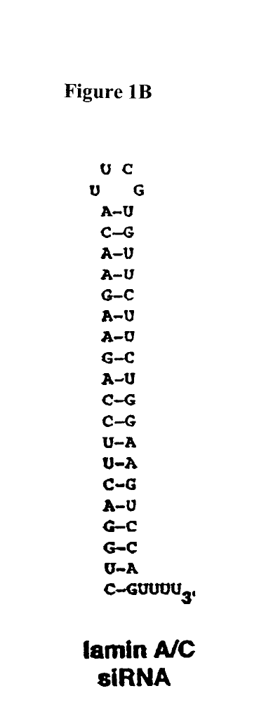

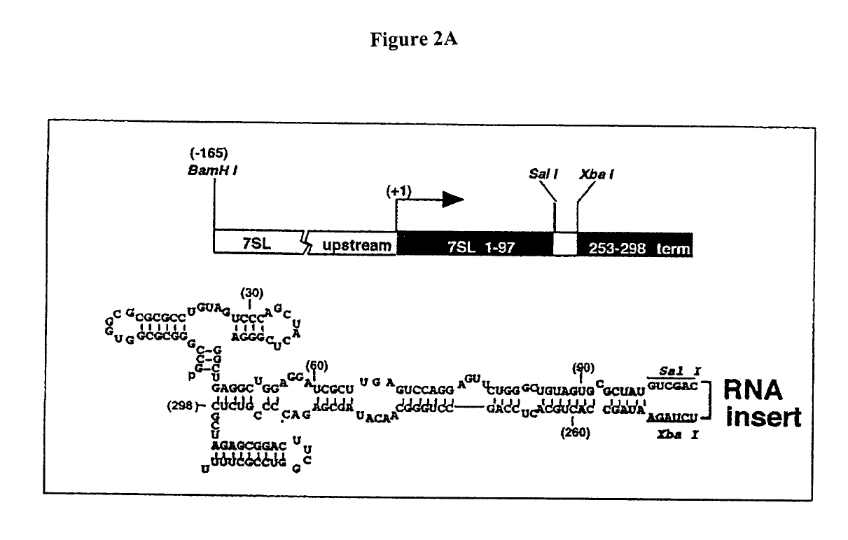

Expression cassettes containing a human U6 promoter and different amounts of U6 RNA coding sequence have been used to drive the expression of small ribozymes, antisense oligoribonucleotides, and RNA aptamers directed against HIV-1 RNA and proteins (Good, P D et al. (1997) Gene Therapy 4: 45-54). These transcripts had strong, artificial stems with stable tetraloops after the insert sites and immediately preceding the polyU terminator for pol III. This structure was expected to protect against 3′-5′ exonuclease attack, the most prevalent small RNA breakdown pathway, and to reduce the chances of the 3′ trailer interfering with the insert RNA folding. The U6 expression cassettes were of 3 types. All had the human U6 gene upstream sequence (Kunkel G R et al. (1986) Proc Natl Acad Sci USA 83:8575-8579; Kunkel G R, and Pederson T. (1989) Nucleic Acids Res 17(18):7371-7379) from positions−1 to −265 and the same 3′ stem at the transcript terminator. The only difference was that the three types of transcripts began with different amounts of U6 RNA. “U6+1” began with +1 as the Sal I cloning site for the insert, thus having no U6 RNA and only a short leader corresponding to the Sal I sequence. “U6+19” had the SalI site inserted after +19, which includes the leading stem of U6. “U6+27” had the SalI site after +27. This construct, shown in FIG. 1A, also includes the full sequence required for 5′γ-phosphomethyl “capping” and stabilization (Noonberg S B et al. (1994) Nucleic Acids Res 22(14):2830-2836; Gupta S, et al. (1990) J Biol Chem 265(31):19137-19142; Shumyatsky G, et al. (1993) Nucleic Acids Res 21(20):4756-4761; Singh R, et al. (1990) Molecular Cell Biol 10(3):939-946). The +19 to +27 sequence was implicated in stability, as well as capping, and the capping was hypothesized to signal nuclear retention. These different constructs were designed to test whether only the +1 to +19 stem improved stability, and whether this RNA would find its way to the cytoplasm without the 5′γ-phosphomethyl cap. The restriction cloning sites between the inserts and the flanking sequences (5′ leaders and 3′ stems) served as structural spacers. Because the human U6 promoter is upstream of the transcription start site, transcripts can be expressed that lack all endogenous U6 RNA sequences. Such sequences were compared to a strategy of including either the first 19 nucleotide U6 RNA stem, or including the first 27 nucleotides, thus also providing the sequence known to be required for U6-specific 5′ capping. As expected, including the first 19 and first 27 nucleotides of U6 significantly increased the levels of the RNA transcripts, with the +27 being both capped and increased threefold over +19. Thus, the steady state levels of RNA increased from U6+1 to U6+19 to U6+27, but the localization of all three transcripts remained similar, which was the nucleus. In all cases, the accumulated transcripts tended to be near full-length, which suggested that if the terminal structures were effectively blocking exonuclease attack and that if internal cleavages occurred, the resulting RNA did not accumulate.

Two unexpected conclusions regarding the RNA expression were derived from the results. The first was that the exact RNA inserted in the cassettes very strongly influenced the level of accumulated RNA in the cell, and in ways that were not necessarily predictable in advance. The second unexpected conclusion was that the default pathway for all RNAs expressed from these promoters appeared to be the nucleus. This suggested that if cytoplasmic localization is needed, it might be necessary to use active cytoplasmic localization signals.

Therefore, two expression cassettes tested in developing the present invention were the U6+1 and the U6+27 gene promoter expression cassettes; these cassettes are described in Example 1 and shown in FIG. 1A. The siRNA inserts transcribed from these expression cassettes are expected to remain in the nucleus; however, it was anticipated that these nuclear transcripts would not be effective in RNAi, and that it would be necessary to develop expression cassettes which might express siRNA which would then be exported to the cytoplasm.

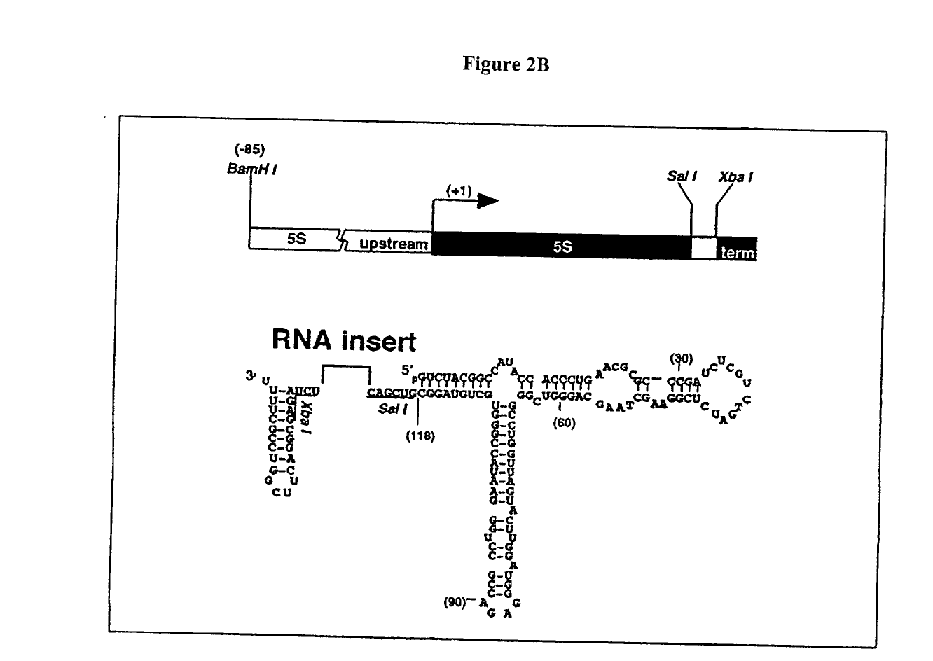

5S rRNA or 7SL signal recognition particle RNA, in contrast to U6 RNA, do appear to be transported to the cytoplasm. The endogenous levels of both 5S and 7SL RNAs are highly abundant in cytoplasm, making moderate production of hybrid 5S and 7SL RNA molecules unlikely to create toxicity by competing for a limiting pool of protein partners. The predicted destination signals are the P9 and P14 protein binding domains, which are composed of a structure between the 5′ and 3′ terminals; these signals have not been published, but are deduced from data which suggests that suggests that their presence results in cytoplasmic transport. Therefore, expression cassettes were designed which employed the promoters of these genes, and which also included the gene sequences which encoded the p9 and p14 binding domains, with everything in between deleted; these expression cassettes are described in Example 1 and shown in FIG. 2. The 5S gene promoter expression cassettes have the entire 5S RNA coding region, so they were expected to assemble normally into RNPs in nucleoli and be exported from the nucleus. The 7SL gene promoter expression cassettes have the early part of the transcripts, which bind the p9/p14 proteins necessary for nuclear export. 7SL transcripts are also thought to transiently associate with nucleoli (Jacobson, M. R. & Pederson, T. (1998) Proc. Natl. Acad. Sci. USA 95, 7981-7986). It was unclear whether the 7SL expression cassette transcripts would also need the 3′ 7SL sequences past the siRNA insert to complete the stem, or whether the leader alone would suffice for eventual cytoplasmic localization. As described below, the leader sequence alone is apparently sufficient, since both the 5S and 7SL transcripts ending with the anti-lamin siRNA are localized primarily in the cytoplasm

In summary, all of the expression cassettes are designed so that a short sequence encoding the RNA to be expressed is inserted between unique Sal I and Xba I sites. All of the cassette transcripts are designed to end with a poly-U termination signal for RNA polymerase III transcription termination, preceded by a strong stem encoded by the cassette to protect the transcripts against 3′-5′ exonuclease attack.

Yet another aspect of the effectiveness of siRNAs is the design of a gene which results in an RNA transcript with the appropriate secondary and tertiary structure; for siRNAs, it is contemplated that an appropriate gene would result in the synthesis of double stranded RNA of the appropriate length with the appropriate overhangs. It is believed that the mediators of sequence-specific mRNA degradation are 21 and 22-nucleotide siRNAs which are generated by ribonuclease III-like cleavage from longer dsRNAs (Elbashir et al. (2001) Nature 411: 494-498). Moreover, base-paired 21-and 22-nucleotide siRNAs with overhanging 3′ ends were reported to efficiently mediate sequence-specific mRNA degradation in lysates prepared from Drosophila embryos, and 21-nucleotide siRNA duplexes with symmetric 2 nucleotide 3′ overhangs were effective in silencing or greatly decreasing the expression of both reporter genes and endogenous genes in mammalian cell cultures (Elbashir et al. (2001) Nature 411: 494-498). However, as noted above, all of these duplexes were synthetic; moreover, most mRNA is transcribed as a single strand, although some transcripts have complementary regions which can fold to form double-stranded segments. Therefore, it was necessary to design genes which would result in transcripts containing dsRNA of the correct length, about 18 to about 25 base pairs in length. From the work with synthetic siRNAs, it was predicted that at least one if not both of the two strands would need an overhang of two nucleotides on the 3′ end.

Several different siRNAs were designed to address the question of effective intracellular expression of siRNA, which is what genes would result in the synthesis of double stranded RNA of the appropriate length with the appropriate overhangs. The initial siRNAs examined were variants of small, double stranded RNAs that were similar to the siRNA inhibitors of lamin A/C published by Elbashir et al (1991). One expected resulting transcript is shown in FIG. 1, which is the anti-lamin A/C siRNA transcript from the U6+1 expression cassette. This transcript contains a 19 base pair dsRNA, with a tetraloop at one end which is unrelated to the target RNAs; the gene for this transcript is inserted in the Sal1 cloning site, resulting in the transcript of a 5′ overhang of the Sal1 cloning site and a 3′ overhang of a T₄ terminator sequence. The tetraloop was predicted to be very stable, as it “tucks” itself into the final transcript structure. However, the resulting siRNA transcript does not possess the two nucleotide overhang at both 3′ ends which was found to work successfully in the synthesized siRNA in mammalian cells. On the other hand, the T₄ terminator sequence overhang at the 3′ end of the antisense strand is predicted to be subject to 3′ to 5′ exonuclease digestion, resulting in a 2 nucleotide overhang for many of the siRNA transcripts. Because the tetraloop siRNA variant does not possess a two nucleotide overhand at both 3′ ends, another variant was designed which possessed a septaloop (or seven nucleotide loop UUAGCCU) in place of the tetraloop (UUCG) as shown in FIG. 1; this septaloop was predicted to be more susceptible to cleavage, resulting in free priming ends on both strands of dsRNA. Y et another variant anti-lamin A/C siRNA gene is designed to contain an entire tRNA sequence in place of the tetraloop shown in FIG. 1; the resulting transcript possesses a tRNA which is predicted to fold into a loop, and not to pair with the 3′ U overhang. The tRNA is also predicted to be subject to cleavage at its 5′ end by RNAse P, which is anticipated to cleave the tRNA at its junction with UU, resulting in a 3′ OH; this would result in a 2 nucleotide UU overhang on the leading strand and a poly U at the other strand. RNAse P is also a nuclear enzyme, and should therefore process the siRNA transcript in the nucleus.

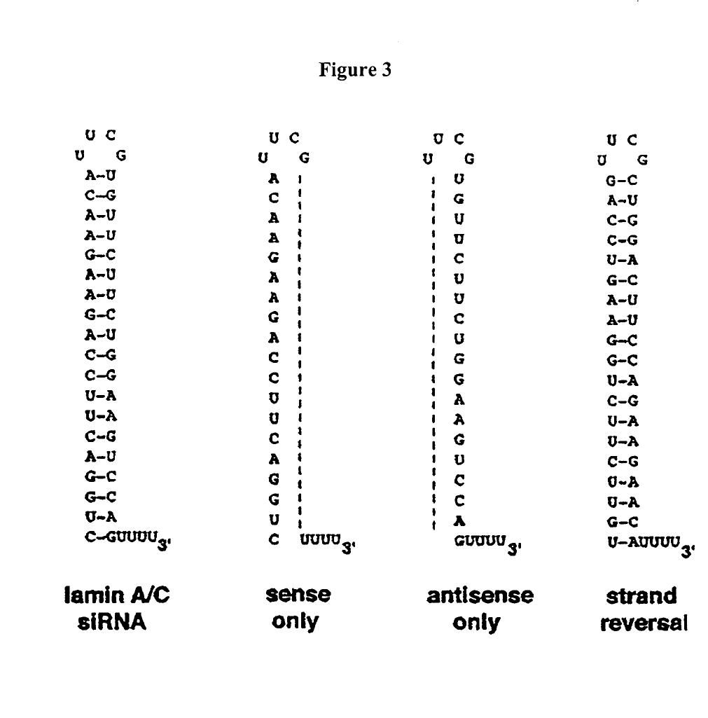

Additional variants of the siRNA genes included several controls, as shown in FIG. 3. These controls included two strands of the anti lamin A/C siRNA expressed separately, where each strand possessed its own tetraloop, and a strand switch, in which the two strands of the anti-lamin siRNA were reversed:

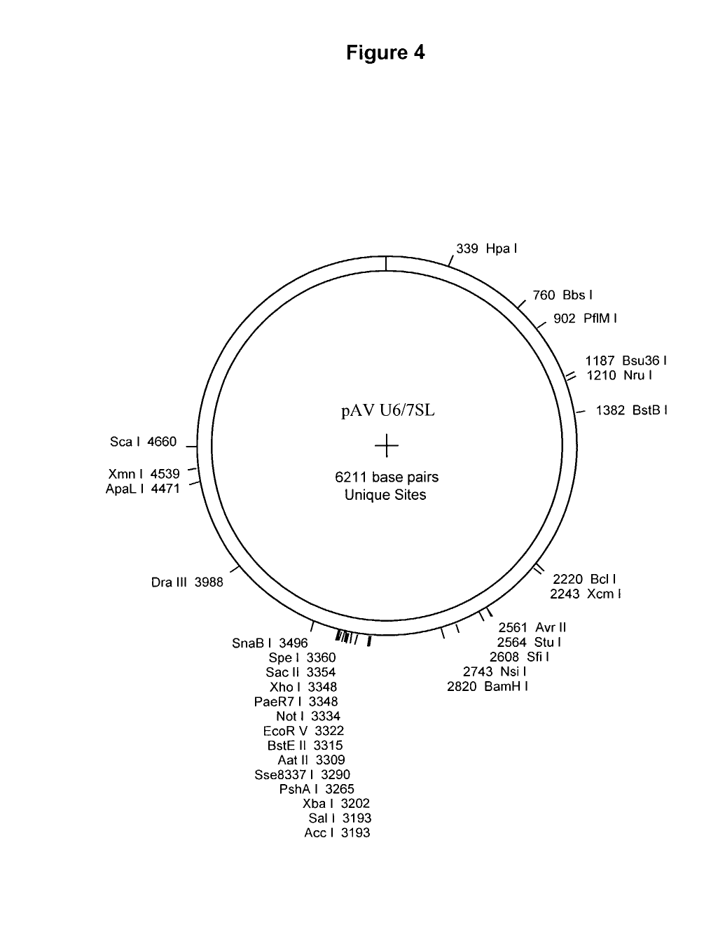

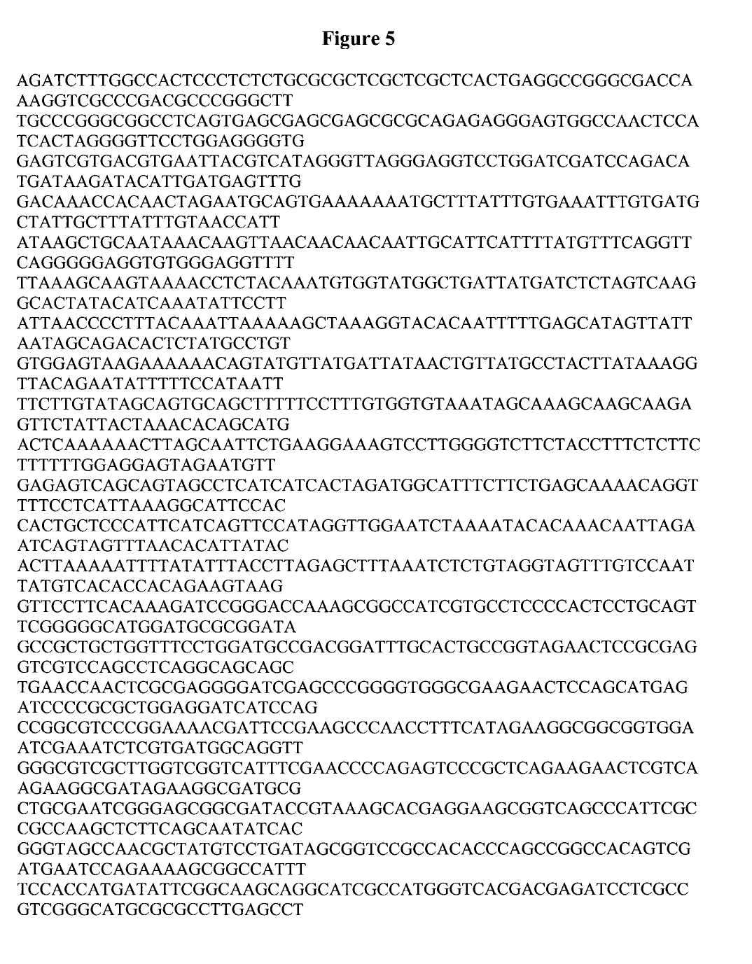

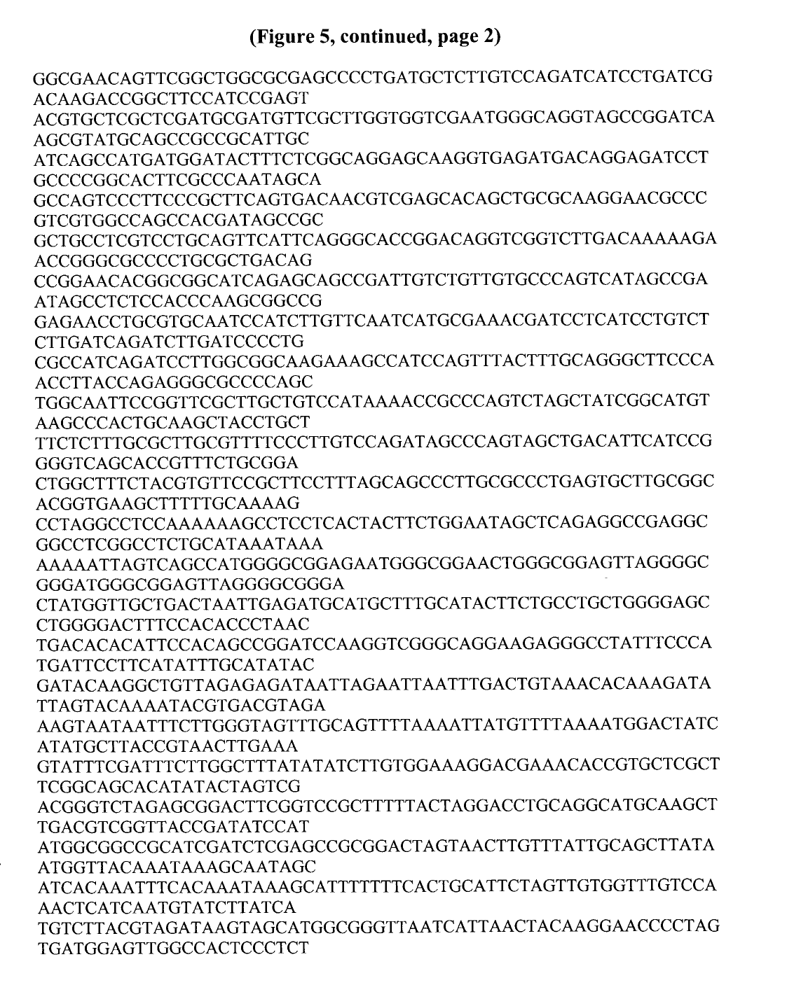

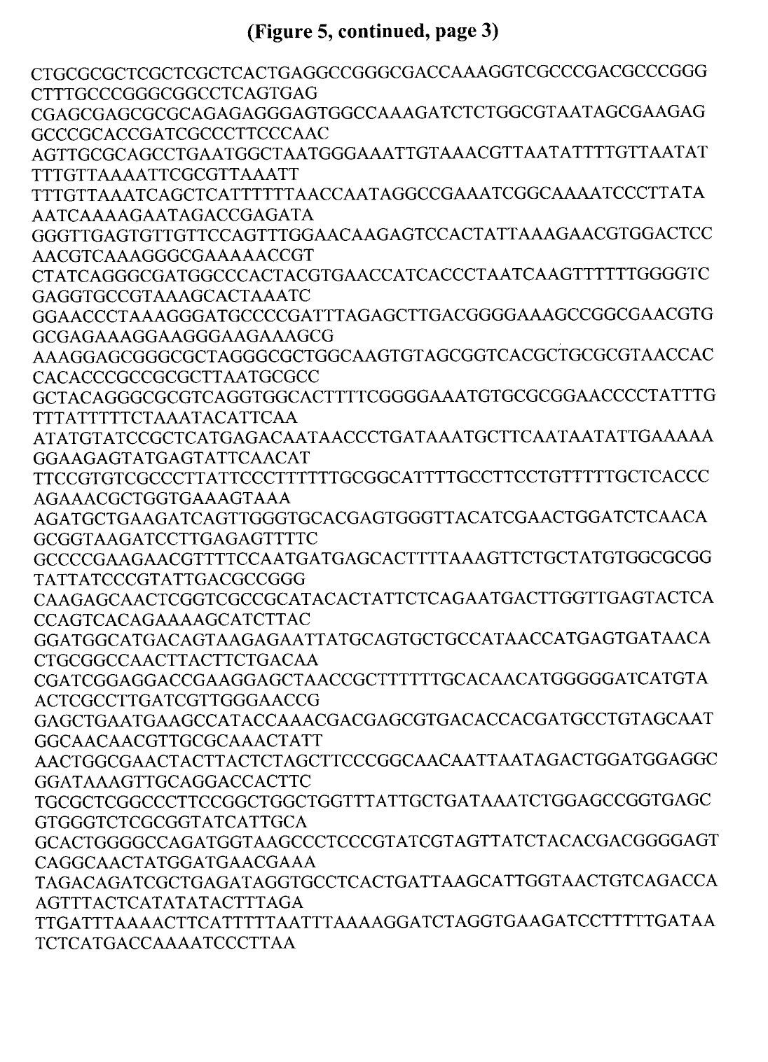

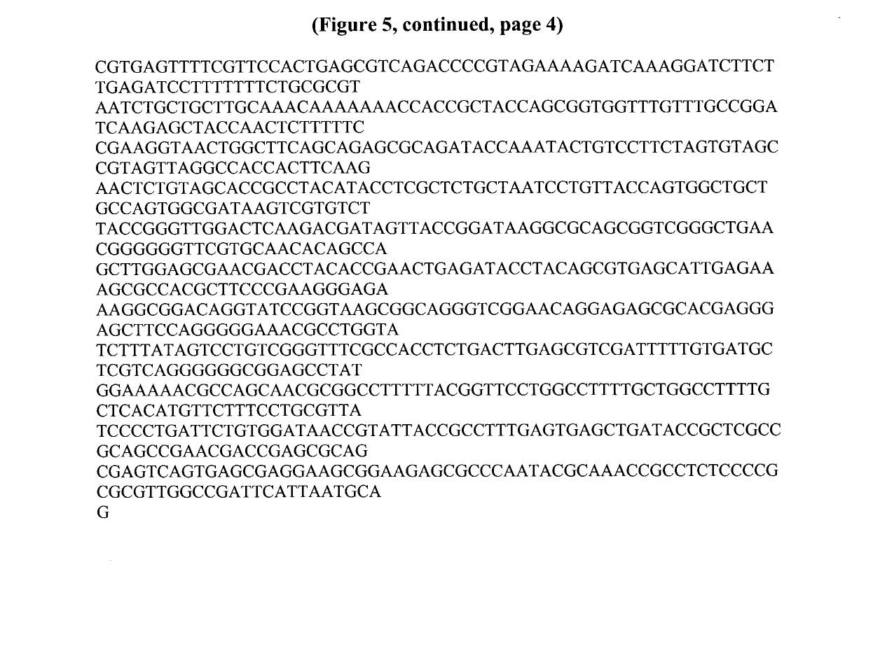

Genes encoding the siRNA variants can then be inserted into a plasmid or vector. A restriction map of the plasmid pTZ18U into which the gene encoding the siRNA variants was inserted is shown in FIG. 4, and the nucleic acid sequence of this plasmid is shown in FIG. 5. The expression cassette was inserted between the BamH1 and the polylinker sites.

The results from the various transcripts described above indicate that transcription of the U6+27 cassette with several inserts, including the lamin siRNA-like insert and the hairpin ribozyme insert, results in the accumulation of the RNA expressed from this cassette in the nucleus, as shown previously (Good, P. D. et al. (1997) Gene Ther. 4: 45-54) and as described in Example 3. Transcription from the U6+27 cassette results in the accumulation of the RNA in nucleoplasmic speckles, similar in distribution to endogenous U6 snRNA. U6+1 transcripts lacking any endogenous RNA sequences and pol III transcripts from tRNA genes, but without tRNA structure, are also nucleoplasmic (Good, P. D. et al. (1997) Gene Ther. 4: 45-54). This leads to the conclusion that pol III transcripts remain in the nucleus by default unless given specific localization signals.

In contrast, RNA transcribed from the 5S and 7SL cassettes accumulates primarily in the cytoplasm, with additional nuclear staining that often corresponds to nucleoli, as defined by DAPI staining, as described in Example 3. For both 5S and 7SL transcripts, the RNA is expected to traffic through the nucleolus before exit to the cytoplasm (Jacobson, M. R. and Pederson, T. (1998) Proc. Natl. Acad. Sci. USA. 95: 7981-7986; Politz, J. C. et al. (2000) Proc. Natl. Acad. Sci. U S A. 97: 55-60). Probes to the endogenous 5S and 7SL sequences suggest that more 5S than 7SL RNA is nucleolar under steady state conditions, but the artificial constructs from both cassettes give bright nucleolar signals in addition to strong cytoplasmic localization. Thus, the 5S and 7SL cassettes can be used when the target is available in the nucleolus or cytoplasm.

Of the various siRNA constructs designed to suppress lamin A/C expression, the expression strategy that was most effective initially was to use variants of the expression cassette derived from a human U6 snRNA gene. These U6 cassettes are described above, and consist of the upstream region of the U6 gene, varying amounts of the U6 snRNA- encoding region, a cloning site, and a terminal stem and TTTT terminator sequence. As noted above, other RNA encoding sequences inserted into the cloning site were demonstrated to be expressed in human cells when the DNA was introduced into cultured human cells; moreover, the bulk of the RNA expressed from the U6 cassettes was retained in the nucleus, regardless of whether part of the U6 snRNA sequence was included as part of the RNA transcripts (Good et al., Gene Therapy 4: 45-54, Bertrand et al., RNA 3: 75-88, 1997). The expression of siRNA was tested in expression cassettes containing either no U6 snRNA sequences (Sal I cloning site replacing the U6+1 sequence, FIG. 1A) or the first 27 nucleotides of the U6 sequence before the Sal I cloning site (named U6+27, FIG. 1A). Variant test siRNA sequences were inserted at the Sal I cloning site; these sequences were followed by a T₄ terminator sequence (leaving 2-4 U residues at the 3′ end of most transcripts). The terminator is followed by another cloning site (XbaI) and a sequence encoding a strong RNA stem and another strong RNA polymerase m terminator.

The effects of transfecting cells with expression cassettes containing anti lamin A/C siRNA variants were compared to those observed with transfecting cells with synthetic anti lamin A/C siRNAs as described by Elbashir et al. (2001) ( Nature 411: 494-498); these experiments are described in Example 3. Cultured human HeLa cells were transfected with either pre-made synthetic siRNA or a combination of β-galactosidase-expressing plasmid and one of three U6 expression cassette plasmids expressing siRNA cassettes (empty U6+1 negative control, U6+1 expressing anti-lamin A/C siRNA, U6+27 expressing anti-lamin A/C siRNA), and examined by fluorescence microscopy. The presence of the β-galactosidase-expressing plasmid allows differentiation of transfected and untransfected cells. Normally, cells that take up one type of plasmid DNA are expected to have taken in a mixture of both co-transfected DNAs.

The results show that in the synthetic transfected siRNA cells, which is a positive control, some cells do not have the characteristic lamin A/C staining (red) around the nucleus (blue); these cells are presumably those that have taken in the siRNA in oligofectamine, resulting in reduced expression of lamin A/C. In the HeLa cells that were co-transfected with plasmids expressing β-galactosidase and one of the U6 expression cassettes, it was consistently observed that cells that were successfully transfected with DNA (as seen by β-galactosidase expression) and an anti lamin A/C siRNA-expressing construct were deficient in lamin A/C. The U6+1 expression cassette without an anti-lamin siRNA has no detectable affect on lamin A/C expression.

The U6 expression cassette was initially designed to result in a strong stem followed by a termination sequence. However, in the U6+27 expression cassette, the stem was effectively removed by the 4 Us which preceded it; the siRNA transcript expressed from this cassette was quite effective, as noted above. In an alternative construct, the strong stem was preceded by 2 U residues, which causes RNA polymerase III to transcribe through the cassette-encoded extra stem and then terminate, preserving the second stem. This construct also worked poorly to suppress lamin A/C, although some reduction in the lamin signal in transfected cells compared to non-transfected cells was consistently observed.

In other experiments, expression cassettes based on 5S rRNA genes or 7SL signal recognition particle RNA genes did not initially effectively express siRNA (as described in Example 3).

RNA blot analysis showed that most of the RNA expressed from these cassettes was near the size expected for full-length transcripts from the indicated +1 positions to the first UUUU terminator. Moreover, the siRNA transcripts made from the U6+1 and the U6+27 expression cassettes were retained in the nuclei, whereas those from the 5S and 7SL expression cassettes were localized in the cytoplasm. This indicates that the 7SL transcripts do not also need the 3′ 7SL sequences past the siRNA insert to complete the stem.

These results, taken together with the results observed for the U6 expression cassettes, are surprising and unexpected. As noted above, it would be reasonable to believe that siRNAs exert their effect cytoplasmically, on target mRNAs. Moreover, the transcript products of the U6 expression cassettes have been shown to be localized to the nucleus, while those expression cassettes based upon 5S rRNA genes or 7SL signal recognition particle RNA genes are believed to be transported to the cytoplasm. Thus, it was believed that only siRNA from the second set of expression cassettes would be effective, while those from the U6 based expression cassettes were less likely to be successful.

Thus, the location in which a particular target will be most accessible is not necessarily predictable in advance. The lamin mRNA target of siRNA is expected to be found primarily in the cytoplasm, but only nuclear delivery of an siRNA-like insert caused a decrease in the lamin signal in transfected cells. While the presence of small amounts of the products from the U6+27 cassette in the cytoplasm cannot be excluded, the vast majority of the hairpin siRNA is in the nucleoplasm (as described in Example 3). The same insert did not reduce lamin levels when expressed from the 5S or 7SL cassettes. These results suggest that the lamin messages (or pre-mRNA) might be more accessible for attack while in the nucleus. The availability of a set of cassettes that provide for different subcellular distributions of a particular RNA insert allows simultaneous testing of an insert in the different cellular compartments.

The observations that siRNA expression is effective in the nucleus, rather than in the cytoplasm as expected, underscores the need to test multiple expression pathways when testing the function of small RNAs inside cells. The development of the 5S and 7SL promoter cassettes for the expression of small RNAs in human cells provides a mechanism for delivering small RNA products to both the nucleolus and cytoplasm. Together with the previously described cassettes based on the U6 snRNA promoter (Good, P. D. et al. (1997) Gene Ther. 4: 45-54) that result in nucleoplasmic localization of their products, this set of cassettes provides for delivery of small RNAs to a variety of cellular compartments. Thus, the U6, 5S, and 7SL expression cassettes described here provide both nucleoplasmic and cytoplasmic delivery capacity. When combined with the previously described nucleolar delivery using similar cassettes (Michienzi, A., et al. (2000). Proc. Natl. Acad. Sci. USA. 97: 8955-8960), these strategies allow testing of a broad spectrum of subcellular destinations. The ability to reach different subcellular locations is contemplated to be important for delivery of diverse small RNAs, depending on the intended targets. For example, a ribozyme targeting HIV-1 mRNA was effective when localized in the cytoplasm, but not when localized to the nucleoplasm (Bertrand, E. et al. (1997) RNA. 3: 75-88). However, as described in Example 3, siRNA transcribed from a U6 expression cassette, which should result in nuclear localization of the transcribed siRNA, was effective in inhibiting co-transfected HIV-1 provirus expression.

Several additional experiments were undertaken with different siRNA variants inserted in a U6+27 expression cassette. In one set of control experiments, when the variant anti-lamin A/C siRNA insert is only one strand of the siRNA as described above, so that each strand of the siRNA is expressed separately, transfection of cultured human cells with either variant siRNA in an expression cassette had no effect on lamin A/C expression. Interestingly, the reverse strand did have some activity, as did the strand without the polyU overhang. In other control experiments, the two strands of the variant anti-lamin siRNA were reversed, as described above, so that the sense strand was followed by the UUUU termination sequence, while the antisense strand was followed by the tetraloop structure and no free 3′ end. Although there was some reduction of the lamin signal with this construct, transfection of cultured human cells with the strand switched variant siRNA in an expression cassette was not as effective as the original orientation, with the antisense strand having the 3′ overhang. Moreover, a less structured seven-nucleotide loop was tested in the siRNA transcript as well; this construct was designed to increase the chances of a nucleolytic cleavage between the strands. However, this siRNA was less effective than the siRNA with a tetraloop.

Additional experiments were undertaken to examine the effects of the overhang from the siRNA. A single overhang from the 3′ end of the antisense strand of the siRNA was quite effective; however, a single overhang from the 3′ sense strand of the siRNA was less effective. Finally, as noted above, U6+27/siRNA construct in was made in which two U residues were removed from the sequence immediately following the siRNA duplex, causing the RNA polymerase III to transcribe through the cassette-encoded extra stem and then terminate. Although this construct also worked poorly to suppress lamin A/C, some reduction in lamin signal in transfected cells compared to non-transfected cells was consistently observed. This might be due to minor amounts of endonuclease-generated breakdown intermediates with free 3′ UU overhangs after the antisense strand of the siRNA duplex.

In addition to providing a means of expressing siRNA-like RNAs against an endogenous target, the U6+27 cassette provides a mechanism for expressing siRNA hairpin inserts against a viral target. For example, an intracellularly expressed siRNA was designed to target a viral gene introduced into the cell on another plasmid along with the siRNA-like construct, as described in Example 3. In this experiment, HIV-1 polymerase mRNA was targeted using hairpin siRNAs expressed from the U6+27 cassette. The siRNA inserts were directed against two different positions in the HIV-1 RNA sequence. The results demonstrated that a construct targeted against one position (2315) consistently inhibited HIV gene expression, by up 90%. In contrast, a second construct, targeted to a different sequence in the same region (position 2568), did not reduce HIV gene expression by greater than abut 50%. These results demonstrate that viral gene expression can be inhibited by intracellularly expressed hairpin siRNAs.

These experiments also indicate that different target sequences for siRNA inhibition may not be equally effective, just as synthetic siRNA sequences vary dramatically in their effects against the same target (Elbashir, S. M. et al. (2001) Nature. 411: 494-498; Elbashir, S. M. et al. (2001) Genes Dev. 15: 188-200). Based upon these results, it is contemplated that several sequences against any gene of interest should be examined when expressing these RNAs within the cell, in order to determine which sequences are most effective.

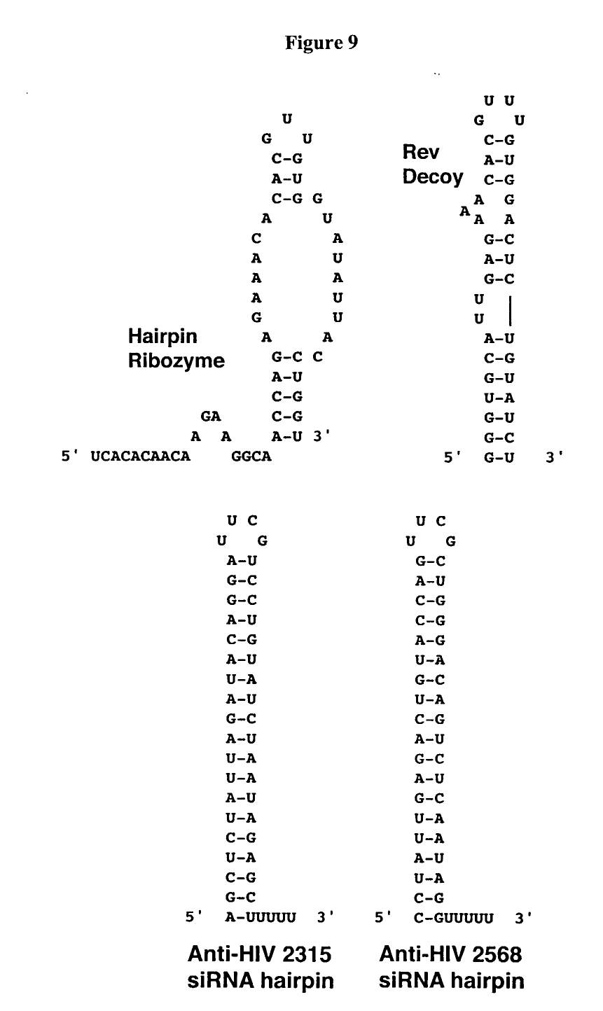

Finally, RNA from U6+27 cassettes with inserts that do not form a long stem loop (hairpin ribozyme and RBE, as shown in FIG. 9) show no evidence of intermediate processing events and no processing of siRNA hairpins was detected. The U6+27 cassette results in a pol III transcript that includes leader sequences in addition to the siRNA that provide added stability. The 5′ 27 nucleotides of the U6 coding sequence are required for modification of the RNA by the addition of a 5′ γ-monomethyl phosphate cap structure and provide protection against attack by exoncleases (Good, P. D. et al. (1997) Gene Ther. 4: 45-54). The absence of processing suggests that a free 5′ end is not required for interference with gene expression by these RNAs.

II. Compositions

A. siRNA

In one aspect of the present invention, the composition comprises a double stranded siRNA of about 18 to about 25 base pairs long, where the double strand is joined at one end by a loop of single stranded RNA; the double stranded region can be as short as 16, and as long as 29, base pairs long, where the length is determined by the antisense strand. Preferably, the RNA is about 19-23 base pairs long, and most preferably, the RNA is about 19 base pairs long. Thus, the present invention provides a composition comprising an siRNA molecule, wherein the molecule comprises a first region, a second region, and a third region, wherein the first region is complementary to and paired to the second region forming a double stranded region such that the double stranded region comprises about 18 to about 25 nucleotide pairs, and wherein the third region links said first region to said second region. The first region or the second region of the siRNA molecule is complementary to a region of a target RNA molecule.

The third, or linking region, is also referred to as a loop. Thus, in some embodiments, the third region is a short loop sequence, from about four to about ten nucleotides in length. The loop may be as small as 3 or 4 nucleotides long; in one embodiment, the loop is 5′UUCG3′. In another embodiment of the present invention, the loop comprises nucleotide sequences which may contain stretches of double stranded RNA as well as stretches of single stranded RNA; in some embodiments, the loop comprises a tRNA. In other embodiments, the third region is a part to all of a tRNA sequence. In some embodiments, the tRNA sequence is modified by additions, deletions, or substitutions of nucleotides.

Preferably, the loop does not interfere with the ability of the siRNA to silence genes. Even more preferably, the loop provides stability, either temporal (as, for example, in preventing degradation) or structural (as, for example, in maintaining a certain configuration, or assisting in binding to RNA or protein). The loop may be subject to processing in vivo, such as cleavage. If the loop is cleaved, it may be cleaved off entirely, or in such a fashion as to leave an overhang, as described below. In other embodiments, the transcript comprises additional sequences of overhanging nucleotides at either the 3′ end or the 5′ end or both ends. Preferably, the nucleotide overhang is about two to five nucleotides; most preferably, the overhang is about two to three nucleotides.

In yet other embodiments, the two strands of the double-stranded region of the siRNA are expressed separately by two different expression cassettes, and then brought together to form a duplex in the cell.

An siRNA may further comprise a destination signal; alternatively, a destination signal is provided by the nature of the promoter. For example, from the U6 promoter described above, unless specific cytoplasmic or nucleolar destination signals are embedded in an RNA transcript, the RNA remains nucleoplasmic. Thus, in some embodiments, the destination signal in the transcription promoter of the RNA product directs the retention of siRNA in the nucleus. Thus, depending upon the promoter, the default localization of RNA transcripts appears to be the nucleus, which means that transcripts without a particular destination signal appear to remain within the nucleus. Other destination signals may be embedded in the RNA transcripts, which include but are not limited to protein binding sites; as examples, the P9 and P14 protein binding sites at the 5′ and 3′ terminals of 5S rRNA and 7SL signal recognition particle RNA are deduced to be cytoplasmic destination signals. Thus, in some embodiments, an siRNA transcript may include the 5′ and 3′ termini of a 5SrRNA or 7SL RP RNA.

An siRNA transcript may comprise additional sequences which confer additional structural stability; such stability may occur as a result of configurational changes, or as a result of preventing degradation. Thus, for example, an siRNA may comprise a strong, artificial stem with a stable tetraloop immediately following the double-stranded stretch and immediately preceding the terminator site for the polymerase (see for example FIG. 1A). This structure is believed to protect against 3′-5′ exonuclease attack, and to reduce the chances of the 3′ trailer interfering with the insert RNA folding. In another example, the first 19 bases of U6 snRNA form a stem loop, which may be included to stabilize the 5′ end of an uncapped siRNA transcript.

An siRNA transcript may comprise additional sequences which result in post-transcriptional modifications. Such modifications include capping the 5′ end of the siRNA transcript. For example, the first 27 nucleotides of the U6 transcript includes the full sequence required for 5′ gamma-phosphomethyl “capping” and stabilization (see for example FIG. 1A). The U6 gene +19 to +27 sequence has been implicated in stability, as well as capping, and the capping has been hypothesized to signal nuclear retention. In designing a gene encoding an siRNA sequence, it is important to avoid sequences that bind to unintended targets. Therefore, the sequence of the siRNA transcript should be specific to the target gene; such specificity is usually achieved by a double-stranded region of about 18 to about 25 base pairs long; the double stranded region can be as short as 16, and as long as 29, base pairs long, where the length is determined by the antisense strand. In particular embodiments, the double-stranded region is about 19-23 nucleotide pairs; in other particular embodiments, the double-stranded region is 19 nucleotide pairs. It has also been observed that the siRNA transcript generally has 100% homology with the target gene, meaning that the transcript is completely homologous to a segment or region of the RNA of the target gene. The strand complementary to a target RNA molecule is the “antisense strand;” the strand homologous to the target RNA molecule is the “sense strand,” and is also complementary to the siRNA antisense strand. One strand of the double stranded region need not be the exact length of the opposite strand; thus, one strand may have at least one fewer nucleotides than the opposite complementary strand, resulting in a “bubble” or at least one unmatched base in the opposite strand. One strand of the double stranded region need not be exactly complementary to the opposite strand; thus, the strand, preferably the sense strand, may have at least one mismatched base-pair.

B. Target Genes

The target of the siRNA may be an endogenous gene, for which the function is either known or unknown, or an exogenous gene, such as a viral or pathogenic gene or a transfected gene. A known gene is one for which the coding sequence is known; the function of such a gene may be known or unknown. Endogenous genes include, for example, disease-causing genes, such as oncogenes, or genetic lesions or defects which result in a disabling conditions. Exogenous genes include reporter genes, marker genes, and selection genes.

Particularly useful reporter genes include, but are not limited to, firefly luciferase, Renilla luciferase, β-gal, green fluorescent protein, chloramphenicol acetyltransferase, β-glucuronidase, alkaline phosphatase, secreted alkaline phosphatase, and human growth hormone. The origin of these genes, their protein characteristics, and the assay for their detection and quantitation are all well known. (See, for example, Current Protocols in Molecular Biology (1995), Chapter 9, “Introduction of DNA into Mammalian Cells,” Section II, “Uses of Fusion Genes in Mammalian Transfection,” (ed: Ausabel, F. M., et al.; John Wiley & Sons, USA), pp. 9.6.1-9.6.12). The latter two proteins are of particular interest, as they are secreted from transfected culture cells into the culture medium. Therefore, the amount of secreted protein can be quantitated from a small sample of the culture medium. However, human growth hormone is not an enzyme, and the protein is therefore be measured directly by an antibody-based assay.

C. Genes; Expression Cassettes

In another aspect of the present inventions, the compositions comprise a gene which encodes at least one siRNA; in related aspects of the present inventions, the compositions comprise an expression cassette comprising a promoter and a gene which encodes at least one siRNA. A gene may encode one siRNA or more than one siRNA.

In some embodiments, the transcribed siRNA forms a double stranded RNA of about 18 to about 25 base pairs long. In other embodiments, the transcribed siRNA forms a double stranded RNA of about 18 to about 25 base pairs long, and further comprises a loop which joins the two strands at one end, as described in any of the embodiments above.

The promoter may be constitutive or inducible; the promoter may also be tissue or organ specific, or specific to a developmental phase. Preferably, the promoter is positioned 5′ to the transcribed region. In one embodiment, the promoter is the U6 gene promoter; in other embodiments, the promoter is a promoter from a 7SL signal recognition particle RNA (srpRNA), or a 5S ribosomal RNA (rRNA). In other embodiments, the promoter (for example, a U6 promoter) is modified so as to possess different specificity. As a non-limiting example, the U6 promoter is modified to a Tet-inducible promoter. In the Tet repressor, the presence of DNA-binding sites interfere with the initiation of transcription from the promoter; thus, the presences of the Tet repressor at the TATA box and other representative sequences result in the U6 promoter being turned off. The addition of tetracycline would thus turn on, or induce, the U6 promoter.

Other promoters are also contemplated; such promoters include other polymerase III promoters, suitably modified as necessary. Thus, exemplary promoters include in addition to the U6 snRNA promoter, tRNA, RNase P RNA, and adenovirus VA RNA pol III promoters as described in the following references, which are hereby incorporated in their entirety (Medina, M. F. C. and Joshi, S. (1999) Curr. Opin. Mol. Ther. 1: 580-594; Brummelkamp, T. R. et al. (2002) Science. 296: 550-553; McManus, M. T. et al. (2002). RNA. 8: 842-850).

Preferably, the expression cassette further comprises a transcription termination signal suitable for use with the promoter; for example, when the promoter is recognized by RNA polymerase III, the termination signal is an RNA polymerase III termination signal. The cassette may also include sites for stable integration into a host cell genome.

D. Vectors

In other aspects of the present invention, the compositions comprise a vector comprising at least one expression cassette; the vectors may further or instead comprise marker genes, reporter genes, selection genes, or genes of interest, such as experimental genes. A vector may also include sites for stable integration into a host cell genome.

In some embodiments of the present invention, vectors include, but are not limited to, chromosomal, nonchromosomal and synthetic DNA sequences (e.g., derivatives of viral DNA such as vaccinia, adenovirus, fowl pox virus, and pseudorabies). It is contemplated that any vector may be used as long as it is expressed and viable in the host; these criteria are sufficient for transient transfection. For stable transfection, the vector is also replicable in the host.

Large numbers of suitable vectors are known to those of skill in the art, and are commercially available. In some preferred embodiments of the present invention, mammalian expression vectors comprise an origin of replication, suitable promoters and enhancers, and also any necessary ribosome binding sites, polyadenylation sites, splice donor and acceptor sites, transcriptional termination sequences, and 5′ flanking non- transcribed sequences. In other embodiments, DNA sequences derived from the SV40 splice, and polyadenylation sites may be used to provide the required non-transcribed genetic elements.

In certain embodiments of the present invention, a gene sequence in the expression vector which is not part of an expression cassette encoding siRNA is operatively linked to an appropriate expression control sequence(s) (promoter) to direct mRNA synthesis. Promoters useful in the present invention include, but are not limited to, the cytomegalovirus (CMV) immediate early, herpes simplex virus (HSV) thymidine kinase, and mouse metallothionein-I promoters and other promoters known to control expression of gene in mammalian cells or their viruses. In other embodiments of the present invention, recombinant expression vectors include origins of replication and selectable markers permitting transformation of the host cell (e.g., dihydrofolate reductase or neomycin resistance for eukaryotic cell culture).

In some embodiments of the present invention, transcription of the DNA encoding a gene is increased by inserting an enhancer sequence into the vector. Enhancers are cis-acting elements of DNA, usually about from 10 to 300 bp that act on a promoter to increase its transcription. Enhancers useful in the present invention include, but are not limited to, a cytomegalovirus early promoter enhancer, the polyoma enhancer on the late side of the replication origin, and adenovirus enhancers.

In other embodiments, the expression vector also contains a ribosome binding site for translation initiation and a transcription terminator. In still other embodiments of the present invention, the vector may also include appropriate sequences for amplifying expression.

Exemplary vectors include, but are not limited to, the following eukaryotic vectors: pWLNEO, pSV2CAT, pOG44, PXT1, pSG (Stratagene) pSVK3, pBPV, pMSG, pSVL (Pharmacia). Particularly preferred plasmids are the Adenovirus vector (AAV; pCWRSV, Chatterjee et al. (1992) Science 258: 1485), a retroviral vector derived from MoMuLV (pGlNa, Zhou et al. (1994) Gene 149: 3-39), and pTZ18U (BioRad, Hercules, CA, USA).

E. Transfected Cells

In yet other aspects of the present invention, the compositions comprise cells transfected by the expression cassettes of the present invention, or by a vector of the present invention, where the vector comprises an expression cassette of the present invention encoding siRNA. In some embodiments of the present invention, the host cell is a mammalian cell. A transfected cell may be a cultured cell or a tissue, organ, or organismal cell. Specific examples of cultured host cells include, but are not limited to, Chinese hamster ovary (CHO) cells, COS-7 lines of monkey kidney fibroblasts, (Gluzman, Cell 23:175 (1981)), 293T, C127, 3T3, HeLa and BHK cell lines. Specific examples of host cells in vivo include tumor tissue.

The cells are transfected transiently or stably; the cells are also transfected with an expression cassette of the present invention, or they are transfected with an expression vector of the present invention. The cells are cultured mammalian cells, preferably human cells, or they are tissue, organ, or organismal cells.

G. Kits

The present invention also provides kits comprising at least one expression cassette encoding at least one siRNA. In some aspects, the expression cassette comprises a promoter operably linked to a gene which encodes an siRNA. In some embodiments, the transcribed siRNA forms a double stranded RNA of about 18 to about 25 base pairs long. In other embodiments, the transcribed siRNA forms a double stranded RNA of about 18 to about 25 base pairs long, and further comprises a loop which joins the two strands at one end, as described in any of the embodiments above. In other embodiments, the expression cassette is contained within a vector, where the vector is used to transfect cells, either transiently or stably.

In other aspects, the kit comprises at least two expression cassettes, each of which encodes one strand of at least one siRNA which, when expressed in a cell, will combine with the strand encoded by the other cassette to form an siRNA; the siRNA so produced is any of the embodiments described above. These cassettes thus comprise a promoter operably linked to a sequence encoding one strand of at least one siRNA. In some further embodiments, the two expression cassettes are present in a single vector; in other embodiments, the two expression cassettes are present in a single vector. The vector is used to transfect cells, either transiently or stably.

III. Methods

The present invention also provides methods of transfecting a mammalian cell with an expression cassette or with a vector as described above. The present invention also provides methods of expressing siRNA in a mammalian cell by transfecting the cell with an expression cassette or with a vector as described above. The present invention also provides methods of silencing a gene in a mammalian cell by transfecting the cell with an expression cassette or with a vector as described above, where the siRNA encoded by the expression cassette targets a gene. In these methods, the cell is transfected transiently or stably, and the cell is a cultured mammalian cell, preferably a human cell, or it is a tissue, organ, or organismal cell. Moreover, in these methods, the target of the siRNA may be an endogenous gene, an exogenous gene, such as a viral or pathogenic gene or a transfected gene, or a gene of unknown function. Furthermore, in the methods the siRNA encoded by the expression cassette or vector upon transcription forms a double stranded RNA of about 18 to about 25 base pairs long. The double stranded RNA may further comprise a loop of single stranded RNA, where the loop joins the double strand at one end; the loop may be as small as 4 nucleotides long. In another embodiments of the present invention, the loop comprises additional nucleotide sequences which may contain stretches of double stranded RNA as well as stretches of single stranded RNA; in some embodiments, the loop comprises a tRNA. The loop may be subject to processing in vivo, such as cleavage.

The compositions of the present invention, including siRNAs, genes encoding at least one siRNA, expression cassettes encoding at least one siRNA, vectors encoding at least one siRNA, and kits comprising expression cassettes or vectors encoding at least one siRNA, are produced by any method well known to the art. Thus, such compositions may be chemically synthesized, or produced by PCR techniques, or produced by cloning.

For example, PCR has been used to express functional U6 cassettes (Castanotto and Rossi (2002) in preparation). If the expression cassette is cloned into a plasmid, large quantities of a particular construct can be grown up and stored for future use. PCR synthesis of siRNAs and expression cassettes has the advantage that many inserts can be tested for function without having to clone them, so it's useful in rapid screening for effective inserts. Moreover, PCR synthesis of siRNAs and expression cassettes also allows easy and quick modification of the inserts, which can then be tested. Because some errors occur with PCR, enzymes which are well known to perform with a reduced error rate are selected for use in PCR. Sequencing of PCR products is also generally not necessary for screening purposes, as a low error rate combined with a large population of molecules means that any error would generally be masked, and the population of PCR-generated products suitable for rapid screening methods.

A. Transfection

In the present invention, cells to be transfected in vitro are typically cultured prior to transfection according to methods which are well known in the art, as for example by the preferred methods as defined by the American Tissue Culture Collection or as described (for example, Morton, H. J., In Vitro 9: 468-469 (1974). When cells to be transfected are in vivo, as in a tissue, organ, or organism, the cells are transfected under conditions appropriate for the specific organ or tissue in vivo; preferably, transfection occurs passively.

Expression cassettes or vectors comprising at least one expression cassette can be introduced into the desired host cells by methods known in the art, including but not limited to transfection, electroporation, microinjection, transduction, cell fusion, DEAE dextran, calcium phosphate precipitation, use of a gene gun, or use of a DNA vector transporter (See e.g., Wu et al., J. Biol. Chem., 267:963 [1992]; Wu and Wu, J. Biol. Chem., 263:14621 [1988]; and Williams et al., Proc. Natl. Acad. Sci. USA 88:2726 [1991]). Receptor-mediated DNA delivery approaches can also be used (Curiel et al., Hum. Gene Ther., 3:147 [1992]; and Wu and Wu, J. Biol. Chem., 262:4429 [1987]).

In some embodiments, various methods are used to enhance transfection of the cells. These methods include but are not limited to osmotic shock, temperature shock, and electroporation, and pressure treatment. In pressure treatment, plated cells are placed in a chamber under a piston, and subjected to increased atmospheric pressures (for example, as described in Mann et al., Proc Natl Acad Sci USA 96: 6411-6 (1999)). Electroporation of the cells in situ following plating may be used to increase transfection efficiency. Plate electrodes are available from BTX/Genetronics for this purpose.

Alternatively, the vector can be introduced in vivo by lipofection. For the past decade, there has been increasing use of liposomes for encapsulation and transfection of nucleic acids in vitro. Synthetic cationic lipids designed to limit the difficulties and dangers encountered with liposome mediated transfection can be used to prepare liposomes for in vivo transfection of a gene encoding a marker (Felgner et. al., Proc. Natl. Acad. Sci. USA 84:7413-7417 [1987]; See also, Mackey, et al., Proc. Natl. Acad. Sci. USA 85:8027-8031 [1988]; Ulmer et al., Science 259:1745-1748 [1993]). The use of cationic lipids may promote encapsulation of negatively charged nucleic acids, and also promote fusion with negatively charged cell membranes (Felgner and Ringold, Science 337:387-388 [1989]). Particularly useful lipid compounds and compositions for transfer of nucleic acids are described in WO95/18863 and WO96/17823, and in U.S. Pat. No. 5,459,127, herein incorporated by reference.

Other molecules are also useful for facilitating transfection of a nucleic acid in vivo, such as a cationic oligopeptide (e.g., WO95/21931), peptides derived from DNA binding proteins (e.g., WO96/25508), or a cationic polymer (e.g., WO95/21931).

It is also possible to introduce an expression or vector in vivo as a naked DNA, either as an expression cassette or as a vector. Methods for formulating and administering naked DNA to mammalian muscle tissue are disclosed in U.S. Pat. Nos. 5,580,859 and 5,589,466, both of which are herein incorporated by reference.

Stable transfection typically utilizes the presence of a selectable marker in the vector used for transfection. Transfected cells are then subjected to a selection procedure; typically, selection involves growing the cells in a toxic substance, such as G418 or Hygromycin B, such that only those cells expressing a transfected marker gene conferring resistance to the toxic substance upon the transfected cell survive and grow. Such selection techniques are well known in the art. Typical selectable markers are well known, and include genes encoding resistance to G418 or hygromycin B.

Although the compositions and methods of the present invention are applicable to situations in which short-term effects of siRNA are to be examined in vitro, such effects can also be observed by adding synthetic siRNA, as has been reported (as, for example, by Elbashir et al. (2001) Nature 411: 494-498). However, in situations in which long-term effects of siRNA are to be examined, it is preferable and in fact necessary to utilize intracellular expression of siRNA. Moreover, it is also necessary to use intracellular expression of siRNA for in vivo effects, as in gene therapy and research applications.

B. Detection of Gene Silencing

The effectiveness of siRNA in a cell can be determined by measuring the degree of gene silencing. Gene silencing can be monitored by a number of means. A “silenced” gene is evidenced by the disappearance of the RNA, or less directly by the disappearance of a protein translated from the RNA. For endogenous genes, rapid protein turnover allows monitoring of gene silencing by protein disappearance; slower protein turnover may be better monitored by measuring mRNA. For exogenous genes, measuring either RNA or protein disappearance would be appropriate.

Detection of the loss of RNA is a more direct measure of gene silencing than is detection of protein disappearance, as it avoids possible artifacts that may be the results of downstream processing. RNA can be detected by Northern blot analysis, ribonuclease protection assays, or RT-PCR. However, measurement of RNA is cumbersome. Therefore, preferred assays measure the presence of a gene protein product.

Proteins can be assayed indirectly by detecting endogenous characteristics, such as enzymatic activity or spectrophotometric characteristics, or directly by using antibody- based assays. Enzymatic assays are generally quite sensitive due to the small amount of enzyme required to generate the products of the reaction. However, endogenous enzyme activity will result in a high background. Antibody-based assays are usually less sensitive, but will detect a gene protein whether it is enzymatically active or not.

IV. Applications

Previous results with siRNA suggest that intracellular expression of siRNA against a wide variety of targets will be effective at reducing or eliminating expression of the targets. For example, an expression cassette can be used in combination with different recombinant DNA vectors to target different cell populations. It is contemplated that either one or more than one expression cassettes are inserted in a vector (the cassettes are relatively small); the siRNA encoded by the expression cassette is directed either to the same target (different stretches of RNA on the same target RNA) or to entirely different targets (e.g. multiple gene products of a virus). It is further contemplated that this method of expressing siRNAs from various expression gene cassettes is useful in both experimental and therapeutic applications. Experimental applications include the use of the compositions and methods of the present invention to the field of reverse genetic analysis of genes found in the human genome sequence. Therapeutic applications include the use of the compositions and methods of the present invention as antiviral agents, antibacterial agents, and as means to silence undesirable genes such as oncogenes.

A. Research Applications

The compositions and methods of the present invention are applicable to the field of reverse genetic analysis, by gene silencing. An siRNA construct can be designed to silence a gene of unknown function, inserted into an expression cassette, and transfected into the cell in which the target gene is expressed. The effect of the lack of or disappearance of an expressed gene product in the transfected cell can then be assessed; such results often lead to elucidation of the function of the gene. Application of siRNA to genes of known function is also contemplated to further examine the effects of the absence of the targeted gene function in a transfected cell.

In some embodiments, research applications are in vitro, as when cultured cells or tissues are transfected with siRNA expression constructs, as described above. In other embodiments, research applications are in vivo, as when organisms such as mammals are transfected with siRNA expression constructs, as described in further detail below.

In some embodiments, the target gene confers a readily perceived phenotype upon the mammal. In these embodiments, an siRNA expression cassette is designed to target the gene for the phenotype. The expression cassette is injected directly into mammalian embryos, and the embryos implanted into a surrogate female parent by well known techniques. Expression of the siRNA gene results in a the phenotype displayed in patterns (because the gene is injected into an embryo, as opposed to a fertilized egg, the result is an individual composed of a mosaic of cells, some of which are transfected with the siRNA gene). The expression of the siRNA gene is confirmed by PCR analysis, and the transgenic mosaic individuals are bred to produce homozygous individuals. This procedure greatly reduces the amount of time required to produce a knock-out line of mammals, which depending upon the mammal, may be decreased by from about fifty percent to ninety percent or more.

B. Therapeutic Applications

The present invention also provides methods and compositions suitable for gene therapy to alter gene expression, production, or function. As described above, the present invention provides compositions comprising expression cassettes comprising a gene encoding an siRNA, and vectors comprising such expression cassettes. The methods described below are generally applicable across many species.

Viral vectors commonly used for in vivo or ex vivo targeting and therapy procedures are DNA-based vectors and retroviral vectors. Methods for constructing and using viral vectors are known in the art (See e.g., Miller and Rosman, BioTech., 7:980-990 [1992]). Preferably, the viral vectors are replication defective, that is, they are unable to replicate autonomously in the target cell. In general, the genome of the replication defective viral vectors that are used within the scope of the present invention lack at least one region that is necessary for the replication of the virus in the infected cell. These regions can either be eliminated (in whole or in part), or be rendered non-functional by any technique known to a person skilled in the art. These techniques include the total removal, substitution (by other sequences, in particular by the inserted nucleic acid), partial deletion or addition of one or more bases to an essential (for replication) region. Such techniques may be performed in vitro (i.e., on the isolated DNA) or in situ, using the techniques of genetic manipulation or by treatment with mutagenic agents.

Preferably, the replication defective virus retains the sequences of its genome that are necessary for encapsidating the viral particles. DNA viral vectors include an attenuated or defective DNA viruses, including, but not limited to, herpes simplex virus (HSV), papillomavirus, Epstein Barr virus (EBV), adenovirus, adeno-associated virus (AAV), and the like. Defective viruses, that entirely or almost entirely lack viral genes, are preferred, as defective virus is not infective after introduction into a cell. Use of defective viral vectors allows for administration to cells in a specific, localized area, without concern that the vector can infect other cells. Thus, a specific tissue can be specifically targeted. Examples of particular vectors include, but are not limited to, a defective herpes virus 1 (HSV1) vector (Kaplitt et al., Mol. Cell. Neurosci., 2:320-330 [1991]), defective herpes virus vector lacking a glycoprotein L gene (See e.g., Patent Publication RD 371005 A), or other defective herpes virus vectors (See e.g., WO 94/21807; and WO 92/05263); an attenuated adenovirus vector, such as the vector described by Stratford-Perricaudet et al. (J. Clin. Invest., 90:626-630 [1992]; See also, La Salle et al., Science 259:988-990 [1993]); and a defective adeno-associated virus vector (Samulski et al., J. Virol., 61:3096-3101 [1987]; Samulski et al., J. Virol., 63:3822-3828 [1989]; and Lebkowski et al., Mol. Cell. Biol., 8:3988-3996 [1988]).

Preferably, for in vivo administration, an appropriate immunosuppressive treatment is employed in conjunction with the viral vector (e.g., adenovirus vector), to avoid immuno-deactivation of the viral vector and transfected cells. For example, immunosuppressive cytokines, such as interleukin- 12 (IL-12), interferon-gamma (IFN-γ), or anti-CD4 antibody, can be administered to block humoral or cellular immune responses to the viral vectors. In addition, it is advantageous to employ a viral vector that is engineered to express a minimal number of antigens.

In one embodiment, the vector is an adenovirus vector. Adenoviruses are eukaryotic DNA viruses that can be modified to efficiently deliver a nucleic acid of the invention to a variety of cell types. Various serotypes of adenovirus exist. Of these serotypes, preference is given, within the scope of the present invention, to type 2 or type 5 human adenoviruses (Ad 2 or Ad 5), or adenoviruses of animal origin (See e.g., WO 94/26914). Those adenoviruses of animal origin that can be used within the scope of the present invention include adenoviruses of canine, bovine, murine (e.g., Mav1, Beard et al., Virol., 75-81 [1990]), ovine, porcine, avian, and simian (e.g., SAV) origin.

Preferably, the replication defective adenoviral vectors of the invention comprise the ITRs, an encapsidation sequence and the nucleic acid of interest. Still more preferably, at least the E1 region of the adenoviral vector is non-functional. The deletion in the El region preferably extends from nucleotides 455 to 3329 in the sequence of the Ad5 adenovirus (PvuII-BglII fragment) or 382 to 3446 (HinfII-Sau3A fragment). Other regions may also be modified, in particular the E3 region (e.g., WO 95/02697), the E2 region (e.g., WO 94/28938), the E4 region (e.g., WO 94/28152, WO 94/12649 and WO 95/02697), or in any of the late genes L1-L5.

In one embodiment, the adenoviral vector has a deletion in the E1 region (Ad 1.0). Examples of E1 -deleted adenoviruses are disclosed in EP 185,573, the contents of which are incorporated herein by reference. In another preferred embodiment, the adenoviral vector has a deletion in the E1 and E4 regions (Ad 3.0). Examples of E1/E4-deleted adenoviruses are disclosed in WO 95/02697 and WO 96/22378. In still another preferred embodiment, the adenoviral vector has a deletion in the E1 region into which the E4 region and the nucleic acid sequence are inserted.

The replication defective recombinant adenoviruses according to the invention can be prepared by any technique known to the person skilled in the art (See e.g., Levrero et al., Gene 101:195 [1991]; EP 185 573; and Graham, EMBO J., 3:2917 [1984]). In particular, they can be prepared by homologous recombination between an adenovirus and a plasmid that carries, inter alia, the DNA sequence of interest. The homologous recombination is accomplished following co-transfection of the adenovirus and plasmid into an appropriate cell line. The cell line that is employed should preferably (i) be transformable by the elements to be used, and (ii) contain the sequences that are able to complement the part of the genome of the replication defective adenovirus, preferably in integrated form in order to avoid the risks of recombination. Examples of cell lines that may be used are the human embryonic kidney cell line 293 (Graham et al., J. Gen. Virol., 36:59 [1977]), which contains the left-hand portion of the genome of an Ad5 adenovirus (12%) integrated into its genome, and cell lines that are able to complement the E1 and E4 functions, as described in applications WO 94/26914 and WO 95/02697. Recombinant adenoviruses are recovered and purified using standard molecular biological techniques that are well known to one of ordinary skill in the art.

The adeno-associated viruses (AAV) are DNA viruses of relatively small size that can integrate, in a stable and site-specific manner, into the genome of the cells that they infect. They are able to infect a wide spectrum of cells without inducing any effects on cellular growth, morphology or differentiation, and they do not appear to be involved in human pathologies. The AAV genome has been cloned, sequenced and characterized. It encompasses approximately 4700 bases and contains an inverted terminal repeat (ITR) region of approximately 145 bases at each end, which serves as an origin of replication for the virus. The remainder of the genome is divided into two essential regions that carry the encapsidation functions: the left-hand part of the genome, that contains the rep gene involved in viral replication and expression of the viral genes; and the right-hand part of the genome, that contains the cap gene encoding the capsid proteins of the virus.

The use of vectors derived from the AAVs for transferring genes in vitro and in vivo has been described (See e.g., WO 91/18088; WO 93/09239; U.S. Pat. No. 4,797,368; U.S. Pat. No., 5,139,941; and EP 488 528, all of which are herein incorporated by reference). These publications describe various AAV-derived constructs in which the rep and/or cap genes are deleted and replaced by a gene of interest, and the use of these constructs for transferring the gene of interest in vitro (into cultured cells) or in vivo (directly into an organism). The replication defective recombinant AAVs according to the invention can be prepared by co-transfecting a plasmid containing the nucleic acid sequence of interest flanked by two AAV inverted terminal repeat (ITR) regions, and a plasmid carrying the AAV encapsidation genes (rep and cap genes), into a cell line that is infected with a human helper virus (for example an adenovirus). The AAV recombinants that are produced are then purified by standard techniques.

In another embodiment, the gene can be introduced in a retroviral vector (e.g., as described in U.S. Pat. Nos. 5,399,346, 4,650,764, 4,980,289 and 5,124,263; all of which are herein incorporated by reference; Mann et al., Cell 33:153 [1983]; Markowitz et al., J. Virol., 62:1120 [1988]; PCT/US95/14575; EP 453242; EP178220; Bernstein et al. Genet. Eng., 7:235 [1985]; McCormick, BioTechnol., 3:689 [1985]; WO 95/07358; and Kuo et al., Blood 82:845 [1993]). The retroviruses are integrating viruses that infect dividing cells. The retrovirus genome includes two LTRs, an encapsidation sequence and three coding regions (gag, pol and env). In recombinant retroviral vectors, the gag, pol and env genes are generally deleted, in whole or in part, and replaced with a heterologous nucleic acid sequence of interest. These vectors can be constructed from different types of retrovirus, such as, HIV, MoMuLV (“murine Moloney leukemia virus” MSV (“murine Moloney sarcoma virus”), HaSV (“Harvey sarcoma virus”); SNV (“spleen necrosis virus”); RSV (“Rous sarcoma virus”) and Friend virus. Defective retroviral vectors are also disclosed in WO 95/02697.

In general, in order to construct recombinant retroviruses containing a nucleic acid sequence, a plasmid is constructed that contains the LTRs, the encapsidation sequence and the coding sequence. This construct is used to transfect a packaging cell line, which cell line is able to supply in trans the retroviral functions that are deficient in the plasmid. In general, the packaging cell lines are thus able to express the gag, pol and env genes. Such packaging cell lines have been described in the prior art, in particular the cell line PA317 (U.S. Pat. No. 4,861,719, herein incorporated by reference), the PsiCRIP cell line (See, WO90/02806), and the GP+envAm-12 cell line (See, WO89/07150). In addition, the recombinant retroviral vectors can contain modifications within the LTRs for suppressing transcriptional activity as well as extensive encapsidation sequences that may include a part of the gag gene (Bender et al., J. Virol., 61:1639 [1987]). Recombinant retroviral vectors are purified by standard techniques known to those having ordinary skill in the art.

In one embodiment, siRNA gene therapy is used to knock out a mutant allele, leaving a wild-type allele intact. This is based on the observation that in order to be effective, the siRNA generally has 100% homology with the sequence of the target gene.

In other embodiments, siRNA gene therapy is used to transfect every cell of an organism, preferably of mammalian livestock.

In yet other embodiments, siRNA therapy is used to inhibit pathogenic genes. Such genes include, for example, bacterial and viral genes; preferred genes are those which are necessary to support growth of the organism and infection of a host. In alternative embodiments, siRNA gene therapy is used to target a host gene which is utilized by a pathogen to infect the host. In some embodiments, the siRNA transcripts are similar to those in FIG. 1B, with a tetraloop, and with a 19 nucleotide pair which is 100% homologous to a specific sequence of the target gene. The siRNA genes are then inserted into an expression cassette, such as is depicted in FIG. 4. This cassette is then placed into an appropriate vector for transient transfection; such vectors are described above. The time course of the transfection is preferably sufficient to prevent infection of the host by the pathogen. The vector is then used to transfect the organism in vivo. In alternative aspects, the vector is used to transfect cells collected from the host in vitro, and the transfected cells are then cultured and re-implanted into the host organism. Such cells include, for example, cells from the immune system.

### EXPERIMENTAL

The following examples are provided in order to demonstrate and further illustrate certain preferred embodiments and aspects of the present invention and are not to be construed as limiting the scope thereof.

In the experimental disclosure which follows, the following abbreviations apply: N (normal); M (molar); mM (millimolar); μM (micromolar); mol (moles); mmol (millimoles); μmol (micromoles); mmol (nanomoles); pmol (picomoles); g (grams); mg (milligrams); μg (micrograms); ng (nanograms); 1 or L (liters); ml (milliliters); μl (microliters); cm (centimeters); mm (millimeters); μm (micrometers); nm (nanometers); ° C. (degrees Centigrade); RNAi (RNA interference); siRNA (short or small interfering RNA)

### EXAMPLES

### Example 1

### Recombinant DNA and RNA Constructs

Expression Cassettes. The general structure of a human U6 snRNA gene promoter expression cassette is shown schematically in FIG. 1A. The cassette contains 265 base pairs upstream of the normal transcription start site. This upstream region contains the transcription regulatory sequences (Kunkel and Pederson, Genes & Devel. 2: 196-204, 1988). A SalI restriction endonuclease site occurs either by replacing the transcription initiation site (+1, referred to as U6+1) or after the first 27 base pairs of the U6 snRNA-encoding region (+27, referred to as U6+27). A second unique cloning site, XbaI, occurs immediately after the SalI site, and allows directional cloning of DNA fragments from the SalI to the XbaI sites. The cassette contains a short region encoding a strong RNA stem immediately following (actually including) the XbaI site. Transcripts are expected to be made from the cassette by RNA polymerase III, which will terminate at UUUU sequence (TTTT in the DNA) at the end of this strong terminal stem. The expected transcripts obtained from U6 expression cassettes are shown below the general schematic; these transcripts are shown without the RNA insert, but the location of the RNA insert is indicated. These transcripts are expressed from either the U+27 expression cassette, or the U+1 expression cassette.

It should be noted that including a sequence encoding 4 or more contiguous Us in the “RNA insert” region will create a transcription terminator before the cassette stem/terminator is reached. It is contemplated that such a termination immediately after the siRNA duplex and before additional sequences can be added and will have positive effects. Minor amounts of read-through to the cassette terminator might occur if the insert contains only four contiguous Us, depending on the surrounding “RNA insert” sequences.

The expected structure of the anti-lamin A/C siRNA transcript from the U6+1 cassette is shown in FIG. 1B. If the transcript were to be from U6+27, the first 27 nucleotides of the human U6 RNA would also be expected (see FIG. 1A). Note that a UUUU sequence has deliberately been inserted at the end of the siRNA double-stranded region. In vivo this is expected to yield a mixture of molecules with 2-4 U residues due to either early pausing or exonuclease trimming of the 3′ end.

The general structures of a human 7SL signal recognition particle RNA gene promoter and 5S rRNA gene promoter expression cassettes are shown schematically in FIGS. 2A and 2B. The 7SL expression cassette contains 165 base pairs of the upstream promoter region, the first 97 base pairs of the 7SL RNA coding region containing the intragenic RNA polymerase III promoters, the Sal I/Xba I cloning sites, the last 48 base pairs of the 7SL sequence (positions 253-301), and the artificial stem/terminator common to all of the cassettes. The structure shown is sufficient to bind two of the signal recognition particle proteins, p9 and p14, and to cause export of the transcript to the cytoplasm, but the signals for interactions with the translation machinery are lost (Strub et al., 1991; Weichenrieder et al., 2001; He et al., 1994). The 5S expression cassette contains 85 bp of upstream sequence and the entirety of the coding sequence before the Sal I/XbaI cloning site replaces the normal terminator. The coding region contains the intragenic promoter and protein interaction signals for export to the cytoplasm.

The human 5S cassette was created by PCR amplifying the 85 nucleotides upstream of the transcription start site through the entire 5S rRNA sequence using the ph5S 8544 plasmid (gift of Beth Moorfield) as template (Moorefield, B. and Roeder, R. G. (1994). J. Biol. Chem. 269: 20857-20865). The oligonucleotides for this PCR were designed with a Bam H1 site on the upstream side and a Sal I site downstream of the 5S rRNA sequence. The fragment was cut and subsequently ligated into pAVU6+27 (Good, P. D. et al. (1997). Gene Ther. 4: 45-54) which was restriction endonuclease treated with the same enzymes as the PCR product.

The 7SL cassette was made by PCR amplifying and subcloning two distinct regions of the p7L30.1 plasmid (Ullu, E. and Wiener, A. M. (1984) EMBO J 3: 3303-3310) (gift of Christian Zwieb). The first fragment amplified contained 7SL RNA sequence from the 258th to the 298th nucleotide of the transcript. The PCR oligonucleotides for the first amplification were designed with an Xba I restriction endonuclease site on the upstream side of the fragment and an Nhe I site on the downstream side. This fragment was ligated into the Xba I site of the pTZU6+1 vector (Good, P. D. et al. (1997). Gene Ther. 4: 45-54). This intermediate vector was cut with Bam H1 and Sal I and used to clone the second PCR product which contained sequence from 165 nucleotides upstream of the 7SL transcription start site to the 97th nucleotide of the 7SL RNA. Oligonucleotides used in this second PCR were engineered with a Bam HI site upstream of the 7SL promoter and a Sal I site downstream of the 97th nucleotide of the 7SL RNA. The resulting expression cassette was later subcloned into pCWRSVN (Chatterjee, S. et al. (1992) Science. 258: 1485-1488) in the same manner as the U6+27 cassette (Good, P. D. et al. (1997). Gene Ther. 4: 45-54).

Inserts were made by annealing oligos of the desired sequence. Sal I and Xba I sites were present near the 5′ and 3′ ends of the annealed oligos, respectively. The annealed oligos and the plasmids carrying the cassettes were cleaved by these enzymes and then ligated. All constructs were sequenced.

Plasmids were amplified in DH5aF′ int or SURE2 (Stratagene, La Jolla, Calif.) cells and purified using Qiagen kits (Valencia, Calif.) followed by extraction with phenol or phenol:chloroform:isoamyl alcohol (25:24:1) and ethanol precipitation.

Sequences inserted between the SalI and XbaI sites in the expression cassettes include a decoy RNA, and siRNAs that target specific genes, as well as variant siRNAs, as described further below.

siRNA transcripts: anti-lamin A/C and variants. The expected structure of one anti-lamin A/C siRNA transcript from the U6+1 gene promoter expression cassette is shown in FIG. 1B; this double stranded siRNA includes a tetraloop. If this transcript were expressed from U6+27, the first 27 nucleotides of the human U6 RNA would also be expected (see FIG. 1A). Note that a UUUU sequence has deliberately been inserted at the end of the siRNA double-stranded region. In vivo this is expected to yield a mixture of molecules with 2-4 U residues due to either early pausing or exonuclease trimming of the 3′ end.

The expected structure of control anti-lamin A/C siRNA transcripts from the U6+1 gene promoter expression cassette are shown in FIG. 3. The first variant contains a tetraloop, and is also shown in FIG. 1B. These controls are variants of this first variant, and include two strands of the anti lamin A/C siRNA expressed separately, where each strand possesses its own tetraloop, and a strand switch, in which the two strands of the anti-lamin siRNA were reversed. If these transcripts were expressed from the U6+27 gene promoter expression cassette, the first 27 nucleotides of the human U6 s RNA would also be expected to be present in the transcript (see FIG. 1A).

Other siRNA transcripts: anti-RBE, anti-HIV. Another hairpin siRNA insert targets Rev binding element (RBE) (as shown in FIG. 9); this anti-RBE insert was designed to serve as a potential decoy to bind Rev protein in infected cells (Jensen, K. B. et al. (1994) J. Mol. Biol. 235: 237-247), thus competing with the virus for available protein. Other hairpin siRNA inserts target the polymerase coding region of HIV-1 RNA. These inserts are made up of a 19 base-pair stem in which the two paired regions are connected by a tetraloop. A pol III transcription termination signal of four or five uridines is located at the 3′ end of the siRNA inserts, before the stem terminator at the 3′ end of the cassette.

Plasmids. A restriction map of the plasmid into which an expression cassette with a gene encoding an siRNA variants was inserted is shown in FIG. 4; the nucleic acid sequence of this plasmid is shown in FIG. 5. The expression cassette is inserted between the BamH1 and the polylinker sites. Thus, each of the expression cassettes were cloned into pTZ1 8U (Bio-Rad) that had been restricted with BamHI and HincII.

### EXAMPLE 2

### Materials and Methods

Materials. Lipofectamine 2000, Lipofectin with Plus reagent, and Oligofectamine were obtained from Invitrogen, as were synthetic DNA oligonucleotides for cloning and probes. Cy3-labeled DNA oligos were obtained from Operon. Synthetic RNA oligonucleotides were obtained from Dharmacon. Anti-lamin A/C monoclonal antibodies were obtained from Santa Cruz Biotechnology (sc-7292, used at 1 μg/ml); rabbit anti-β-galactosidase antibodies were obtained from Molecular Probes (A-1132, 1 μg/ml); Oregon green 488-labeled goat anti-rabbit secondary antibodies were obtained from molecular Probes (O-11038, 5 μg/ml) and Cy3-labeled goat antimouse secondary antibodies were obtained from Amersham-Pharmacia Biotech (PA 43002, 1 μg/ml). The small RNA expression cassettes were cloned the pAV vector, derived from pRCRWRSPN (Chatterjee, S et al (1992) Science 258, 1485-1488) by deleting the RSV promoter expression module between a BamH I and unique Xho I sites and substituting a the expression cassettes, followed by a synthetic polylinker. The plasmids retain the SV40 promoter-driven neomycin resistance gene and an ampicillin resistance gene originally derived from pTZ18R (Promega). Sequences to be expressed were inserted using synthetic oligonucleotides precisely between the end of the unique Sal I site and the beginning of the unique Xba I site. All recombinant constructs were sequenced.

Transfection of Cells with siRNA. HeLa cells (obtained from Imperiale lab) and 293 cells were grown in DMEM (Invitrogen, Carlsbad, Calif.) supplemented with 10% fetal bovine serum (HyClone, Logan, UT), 100 units/ml penicillin, 100 mg/ml streptomycin, and 4 mM L-glutamine. Cells were maintained at 37° C. in 5% carbon dioxide by splitting twice each week.

HeLa cells were transfected with DNA constructs using Lipofectamine 2000 or Lipofectin with Plus reagent (Invitrogen). Transient transfections were performed on subconfluent HeLa cells. Synthetic siRNA was transfected using Oligofectamine (Invitrogen) as described, and recombinant DNA constructs were transfected using Lipofectamine 2000 or Lipofectin with Plus reagent generally according to the manufacturer's instructions (Evans, K. et al. (999) Focus. 21: 15).

When transfecting using Lipofectin, cells were plated one day in advance in media lacking antibiotics so that they would be approximately 50% confluent at the time of transfection. For 60 mm plates, 2.8-3.3 μl Lipofectin and 20 μl Plus reagent were used. Plasmids were transfected into cells at a 3:1 ratio of experimental plasmid to pCMVβ (Clonetech, Palo Alto, Calif.; described further below) so that a total of 4.4 pg DNA was added to each plate. The amounts of Lipofectin and DNA were adjusted in proportion to the surface area when the size of the plates was varied. One day after transfection, cells were trypsinized, resuspended in DMEM with 10% fetal bovine serum and L-glutamine, but without antibiotics, and split. Cells to be used for microscopy were plated onto coverslips. Transfection efficiency was determined by cytochemical assay for β-galactosidase expression 3 days after transfection (Sanes, J. R. et al. (1986) EMBO J. 5: 3133-3142).

When transfecting using Lipofectamine 2000, cells were plated one day in advance in 6-well dishes so that they would be 90-95% confluent on the day of transfection. A total of 3.8 mg of DNA was used per well, in a 3:1 molar ratio of experimental plasmid to pCMVb. Following transfection, cells were treated in the same way as described for Lipofectin transfection.

293 cells were transfected using calcium phosphate as described in Good et al. (Good, P. D. et al. (1997) Gene Ther. 4: 45-54). Cells were plated at 5-10% confluency in 100 mm plates containing coverslips 1-2 hours prior to transfection. A total of 10 μg DNA was added to each 100 mm plate in a 3:1 molar ratio of experimental plasmid to pCMVb. The media was not changed following transfection when the cells were plated on to coverslips. Transfection efficiency was assayed 2 days after transfection as described for HeLa cells. pCMVβ, a plasmid containing the gene for β-galactosidase, was co-transfected with the plasmids encoding siRNA expressed from RNA polymerase III promoters. For microscopy, cells were grown on coverslips and fixed approximately 72 hours (day 3) after transfection. Transfection efficiency was estimated by β-galactosidase activity staining of permeablized cells in parallel transfections. In transient transfections, cells were split after one day and allowed to grow for two additional days before examination by fluorescent microscopy.

Fluorescence microscopy. Transfected cells were subjected to different protocols for examining proteins (lamin A/C and β-galactosidase) or the distribution of small, recombinant siRNAs. Detection of lamin A/C and β-galactosidase proteins was performed essentially as described (Elbashir, S. M. et al. (2001) Nature 411, 494-498). Cell permeabilization, annealing of Cy3-labeled oligos was also as described (Good, P. D. et al. (1997) Gene Therapy 4, 45-54). Fluorescence signal was acquired on a Nikon Eclipse E800 microscope with a Hamamatsu Orca II digital camera. Multiple low-resolution fields were examined to determine whether transfected cells lost lamin signal, and whether high resolution images of siRNA localizations were representative. High-resolution images from successive optical planes were deconvoluted using Isee analytical imaging software (Inovision Corp.).

Immunolocalization of lamin A/C. For Immunolocalization of lamin A/C, cells were fixed in methanol for 6 minutes at−20° C. Transfected cells were identified by the detection of β-galactosidase (expressed from a plasmid that was co-transfected with the plasmid expressing the anti-lamin A/C plasmid) using anti-β-galactosidase polyclonal antibodies at 1.0 μg/ml (Molecular Probes A-11132) and Oregon Green 488 labeled goat anti-rabbit antibodies at 5 μg/ml (Molecular Probes, O-11038). Monoclonal antibodies against lamin A/C were from Santa Cruz Biotechnology (lamin A/C (636):sc-7292) and were used at 1 μg/ml. The Cy3-labelled goat antimouse secondary antibodies used to detect lamin A/C were from Amersham Pharmacia Biotech (PA 43002) and were used at 1 μg/ml. Primary and secondary antibodies were diluted in PBS containing 3% BSA and were incubated with the coverslips for 1-2 hours at 37° C. Cells were stained with DAPI (2 g/μl) for 5 minutes. All washes were in PBS. Coverslips were mounted on slides in Prolong (Molecular Probes).

Slides were photographed as described above.

Immunoassay for HIV gene expression. A modified calcium phosphate co-transfection method (Wigler, M. et al. (1978) Cell. 14: 725-731) was used to transfect 293 cells (NIH) for the HIV inhibition assay. Cells were plated in six-well polystyrene tissue culture plates (5×105 cells/well) in 2 ml EMEM (Quality Biological Inc., Gaithersburg, M D) supplemented with 10% FBS (Gemini Bio-Products Inc., Woodland Calif.) and grown for 24 hours. DNA was added at either a 5:1 ratio (0.8 mg:0.2 mg) or a 10:1 ratio (3.6 mg:0.4 mg) of anti-HIV agent to pNL4-3, the plasmid carrying the proviral sequence. DNAs were mixed in 96-well polystyrene tissue culture plates and the volume adjusted to 83 ml with TET9/10 (1 mM Tris, pH 7.9, 0.1 mM EDTA). After the thorough mixing of 13 μl 2M calcium chloride and 100 μl 2×HBS with the DNA in each well, the solutions were incubated for 30 minutes at room temperature then added to the cell cultures. The cultures were incubated at 37° C., medium was changed after 4 hours, and supernatant was collected for the p24 assay daily for four days. Samples of the supernatant culture fluids were tested in a p24(HIV-1) antigen assay using ELISA (enzyme-linked immunosorbent assays) commercial kits (Beckman Coulter, Hialeah, Fla.) according to the manufacturer's recommendations.

Localization of RNA products of siRNA constructs. Cells were fixed on coverslips directly in growth media by adding fixative to bring the concentration to 4% paraformaldehyde (EMS Sciences, Ft. Washington, Pa.) and 10% acetic acid two days after transfection. After fixation cells were permeabilized by submersion in 70% ethanol and storage at 4° C.

Cassette transcripts were localized using oligos tagged with either Cy3 or Oregon Green 488-labeled antibodies, etc, as described below. Hybridization to oligonucleotide probes was in 10% dextran sulfate, 2 mM vanadyl ribonucleoside complex, 0.02% BSA, 40 micrograms yeast tRNA, 2SSC, 50% formamide, and 30 nanograms of probe (http://singerlab.accom.yu.edu/protocols). Following hybridization overnight at 37° C., coverslips were washed twice at 37° C. for 30 minutes each time in 2×SSC, 50% formamide and once in PBS (58 mM Na₂HPO₄, 17 mM NaH₂PO₄, 68 mM NaCl, pH 7.4) for 15 minutes. Cells were stained with 4′,6-diamidino 2-phenlindole (DAPI) at 2 ng/μl in PBS for 5 minutes and washed with PBS 3 times for 5 minutes each time. Coverslips were affixed to slides in Prolong mounting medium (Molecular Probes, Eugene, OR). Slides were viewed and photographed with a Nikon (Melville, N.Y.) Eclipse E800 microscope and a Hamamatsu (Bridgewater, N.J.) Orca II digital camera. Images were subjected to deconvolution using Isee Analytical Imaging Software (Inovision Corporation, Raleigh, N.C.).

Fluorescent probes for in situ localization of siRNA and controls:

Lamin oligo:

| | |
|---|---|
| Cy3 | CGAATGTTCTTCTGGAAGTCCAGGTCGAC |

Endo 5S OG:

| | |
|---|---|
| ORG 488 | CTTAGCTTCCGAGATCAGACGAGATCGGGCGCG |

Endo U3 OG:

| | |
|---|---|
| ORG 488 | CCTCTCTTCCTCGTGGTTTTCGGTGCTCTACACGTTCAGAG |

Endo 7SL OG:

| | |
|---|---|
| ORG 488 | CCGGGAGGTCACCATATTGATGCCGAACTTAGTGCG |

Cy3-labelled oligos were obtained from Operon and Oregon Green 488-labelled antibodies were obtained from Molecular Probes.

Detection of transcripts by Northern blotting. RNA was isolated from transfected 293 cells using Trizol reagent (Invitrogen, Carlsbad, Calif.) 2 days after transfection (FIG. 4 A and B). HeLa cells were transfected as described with experimental plasmids and pEGFP (Clonetech, Palo Alto, Calif.) in an equimolar ratio. The media was replaced 24 hours after transfection. HeLa cells were subjected to cell sorting by the University of Michigan Flow Cytometry Core using a BD FACS Vantage SE (BD Biosciences, San Jose, Calif.) 2 days after transfection. RNA was isolated from sorted HeLa cells using Trizol reagent according to the manufacturer's instructions (FIG. 4C).

RNA samples and size markers were separated on 6% (FIG. 4A and B) or 8% (FIG. 4C) polyacrylamide gels containing 8.3 M urea. The RNA was then blotted to Nytran (Schleicher and Schull, Keene, NH) using the Genie electroblotter (Idea Scientific, Minneapolis, MN) under conditions specified by the manufacturer. The RNA was crosslinked to the Nytran membrane using UV light by placing each side on a transilluminator (Fotodyne Model 3-3000, Hartland, Wis.) for 2 minutes. Oligodeoxynucleotide probes (Invitrogen, Carlsbad, Calif.) that recognize insert RNA were labeled using polynucleotide kinase (New England Biolabs, Beverly, Mass.) and γ-[³²P] ATP (NEN Life Science Products, Boston, Mass.). Separate probes were prepared that hybridize to the RBE insert (5′AGATACAGAGTCCACAAACGTGTTTCTCAATGCACCC 3′), the U6+27 cassette (5′GTCGACTAGTATATGTGCTGCCGAAGCAGCAC 3′), and endogenous human U6 snRNA (5′CACGAATTTGCGTGTCATCCTTGCGCAGGGGCC 3′). The U6+27 and endogenous U6 probes are specific, since the probe for the endogenous U6 snRNA does not hybridize to the U6 sequences included in the U6+27 cassette and the U6+27 probe does not recognize endogenous U6 snRNA under the hybridization and wash conditions used. RNA blots underwent a 1 hour prehybridization in: 6×SSPE buffer (20X=3M NaCl, 200 mM NaH2PO4.H2O, 20 mM EDTA, pH7.4), 200 μg/mL salmon testis DNA (Sigma, St. Louis, N.J.), 1% SDS, 5×Denhardt's reagent (USB, Cleveland, OH). Hybridization was performed by adding labeled oligo probes in: 6×SSPE, 1% SDS. The U6+27 probe was hybridized to the membrane for 2 hours at 70° C., followed by 3 hours at 65° C. All other probes were allowed to hybridize overnight at 68° C. Blots were washed three times with 250 mL of wash buffer (6×SSPE, 1% SDS) for 30 minutes at room temperature, followed by one 250 ml wash for 3 minutes at 65° C. for the U6+27 probe and 68° C. for all other probes. A final 250 ml wash in 6×SSPE was done at room temperature for 30 minutes. The membrane was then exposed to a storage phosphor screen that was subsequently scanned on the Molecular Dynamics Phosphorlmager 445 SI (Piscataway, N.J.). Image analysis, for quantification of hybridization signals, was carried out using IP Lab Gel Software (Signal Analytic Corporation, Vienna, Va.). Expressed RNA copy number was estimated relative to endogenous U6 snRNA in each sample (400,000copies/cell) (Weinberg, R. A. and Penman, S. (1968) J. Mol Biol. 38: 289-304) normalizing for approximate transfection efficiency measured by cytochemical detection of β-galactosidase expression in parallel co-transfections with pCMVβ.

### EXAMPLE 3

### Transfection of Human Cells with siRNA

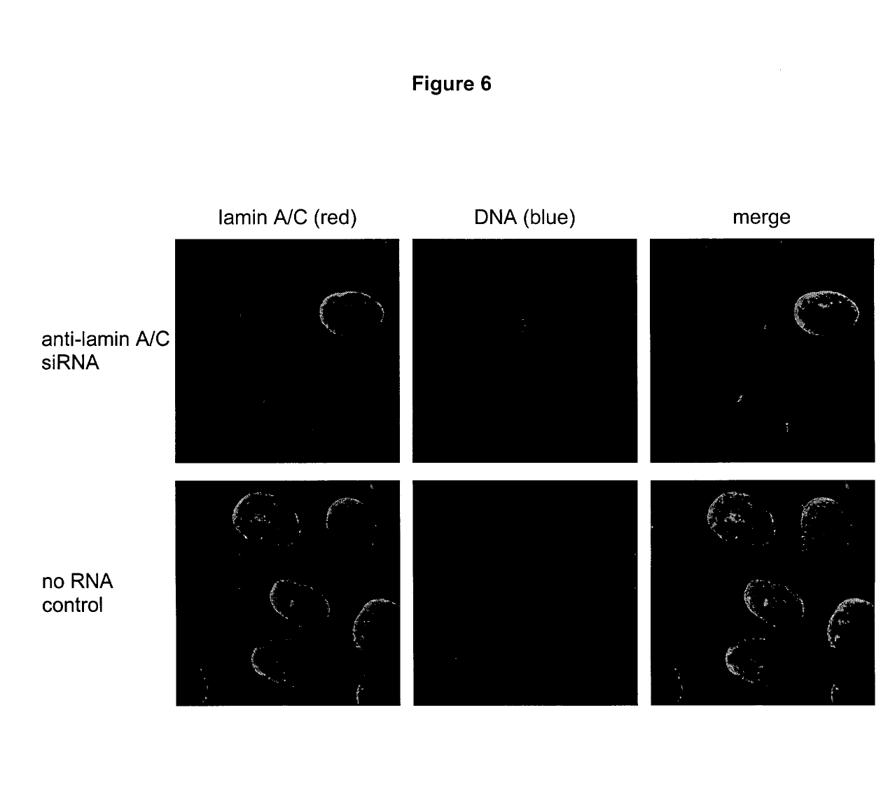

Synthetic anti lamin A/C siRNA. Lamin A/C expression was inhibited by treatment of human HeLa cells with synthetic siRNA. HeLa cells were treated with pre-made or synthetic “siRNA” directed against lamin A/C, using oligofectamine for transient transfection, as described previously (Elbashir et al., Nature 411: 494-498, 2001). The cells were stained with monoclonal antibody against lamin A/C (red signal, Elbashir et al., ibid) or DAPI to visualize the nuclear DNA (blue signal). The results are shown in FIG. 6.

These results show that in the synthetic transfected siRNA cells, which is a positive control, some cells do not have the characteristic lamin A/C staining (red) around the nucleus (blue); these cells are presumably those that have taken in the siRNA in oligofectamine, resulting in reduced expression of lamin A/C. As expected, only some cells are successfully transfected; in the absence of siRNA, all cells show lamin signal.

Intracellularly Expressed siRNAs.

1. Subcellular localization of siRNAs from different pol III expression cassettes. The subcellular localization of siRNAs transcribed from pol III expression cassettes is dependent upon the expression cassettes. This conclusion is drawn from observations of the subcellular localization of siRNAs transcribed from U6, 5S, and 7SL cassettes.

Transcripts were detected in human 293 cells by in situ hybridization of Cy3-tagged oligonucleotides that hybridize to the hairpin ribozyme insert (red). The location of nuclei is indicated by DAPI staining (blue). Nuclear localization is observed when the RNA is expressed from the U6 promoter, as previously demonstrated (Good, P. D. et al. (1997) Gene Ther. 4: 45-54). Transcripts expressed from the 5S promoter are found in the cytoplasm and in spots within the nucleus that correspond to the nucleoli as suggested by DAPI staining. Transcripts expressed from the 7SL promoter are found in the cytoplasm and in spots in the nucleus that overlap with the nucleoli. Endogenous U6, 5S, and 7SL RNAs were detected with Cy3-labelled probes in untransfected cells. The distribution of hairpin ribozyme transcripts is similar to the pattern observed for the corresponding endogenous RNAs.

The results described above demonstrate that transcription of the U6+27 cassette with several inserts, including the anti-lamin siRNA insert, results in the accumulation of the RNA expressed from this cassette in the nucleus. Transcription from the U6+27 cassette results in the accumulation of the RNA in nucleoplasmic speckles, similar in distribution to endogenous U6 snRNA. U6+1 transcripts lacking any endogenous RNA sequences and pol III transcripts from tRNA genes, but without tRNA structure, are also nucleoplasmic. These results indicate that pol III transcripts remain in the nucleus by default unless given specific localization signals.

In contrast, the results demonstrate that RNA transcribed from the 5S and 7SL cassettes accumulates primarily in the cytoplasm, with additional nuclear staining that often corresponds to nucleoli, as defined by DAPI staining. For both 5S and 7SL transcripts, the RNA is expected to traffic through the nucleolus before exit to the cytoplasm (Jacobson, M. R. and Pederson, T. (1998) Proc. Natl. Acad. Sci. USA. 95: 7981-7986; Politz, J. C. et al. (2000) Proc. Natl. Acad. Sci. U S A. 97: 55-60). Probes to the endogenous 5S and 7SL sequences suggest that more 5S than 7SL RNA is nucleolar under steady state conditions, but the artificial constructs from both cassettes give bright nucleolar signals in addition to strong cytoplasmic localization. Thus, the 5S and 7SL cassettes can be used when the target is available in the nucleolus or cytoplasm.

2. Expression of RNA from pol III cassettes in human cells. RNA expressed from the pol III cassettes accumulates to high levels in human cells. This was determined by experiments in which RNA was isolated from cells that had been transfected with plasmids containing the pol III cassettes, and then subjected to polyacrylamide gel electrophoresis, transferred to membrane, and probed with ³²P-labeled DNA oligomers that hybridize to the insert and to endogenous U6 snRNA or to the first 27 bases of U6 snRNA. The plasmids included those expressing a hairpin ribozyme insert expressed from the U6+27, 5S, and 7SL cassettes; an RBE decoy expressed from the U6+27, 5S, and 7SL cassettes; and anti-lamin A/C siRNA inserts expressed from the U6+27 cassette. Lanes containing RNA from mock-transfected cells or cells transfected with pCMVβ alone are included as controls

The results demonstrated that RNA of the expected sizes for full-length transcripts accumulated in cells transfected with the U6+27, 5S, and 7SL cassettes containing the hairpin ribozyme insert and an RBE insert. These results are consistent with earlier observations for antisense, decoy, and ribozyme inserts in cassettes driven by tRNA and U6 pol III promoters (Good, P. D. et al. (1997) Gene Ther. 4: 45-54). It was previously shown (Good, P. D. et al. (1997) Gene Ther. 4: 45-54) that transcription initiated at the expected sites and gave transcripts of the expected full-length, but with some exonuclease trimming of 1-4 U residues from the 3′ end of the cassette-encoded stem terminator. Transcripts expressed from the U6+27 cassette have a γ-monomethyl phosphate cap (Good, P. D. et al. (1997) Gene Ther. 4: 45-54). RNA from all cassettes was generally present at 104-106 copies per cell when normalized to the level of endogenous U6 RNA (Weinberg, R. A. and Penman, S. (1968) J Mol. Biol. 38: 289-304), although there was variability between transfections. Transcripts expressed from the 7SL promoter generally accumulate to at least as high a level as those from the strong U6 promoter and resulted in even greater levels of expression in some experiments. In contrast, the 5S promoter results in a lower level of transcript accumulation in cells than the U6 promoter.

In addition to the strong transcription terminator provided by the cassette-encoded stem-loop and five uridine residues at the 3′ end of all cassettes, cassettes that contain anti-lamin siRNA inserts have four uridine residues immediately after the insert (FIG. 2). The “antisense only” U6+27 control construct contains only the antisense strand from the siRNA construct, along with the loop and U₄ terminator sequences. Transcription of this construct in HeLa cells sometimes terminates at the first U₄ sequence after the insert (25%), but a substantial amount reads through to the cassette-encoded stem-terminator (75%). This termination inefficiency was unexpected, given that U₄ is normally sufficient to terminate vertebrate pol III transcripts (White, R. J. (1998) RNA polymerase III transcription. R. G. Landis, Georgetown), but indicates that at least five U residues are preferably used to definitively terminate transcripts in this system. Both termination sites result in products that are 1-4 nucleotides shorter than predicted from the sequence. The exact number of Us remaining on the 3′ end is variable, presumably due to heterogeneous post-transcriptional trimming by endo- and exonuclease activities, as is commonly seen in pol III products.

An unusual pattern was observed when constructs containing the 19 base pair stem loop of the anti-lamin siRNA (forward or reverse orientation) were expressed in HeLa cells. Small amounts of transcripts are the appropriate length for termination of transcription to have occurred at the fourth U in the U₄ terminator immediately following the siRNA insert, but most of the transcripts are found in four or five shorter bands, each 1-3 nucleotides shorter than the preceding band. This pattern was not observed with any other types of RNA inserts. The shortened RNAs might be the work of nucleases after transcription, but might also result from pol III itself chewing back from the terminator in 1-3 nucleotide bites. Pol III, like other RNA polymerases, is known to have this propensity at strong pause sites (Whitehall, S. K. et al. (1994) J Biol. Chem. 269: 2299-2306). It is possible that the especially strong 19 bp siRNA stems act as strong transcription pause sites to encourage this trimming. Whatever mechanism creates these products appears to require the U₄ terminator after the strong stem, since removing two of the Us causes read-through to the cassette stem terminator, eliminating the strong ladder of bands.

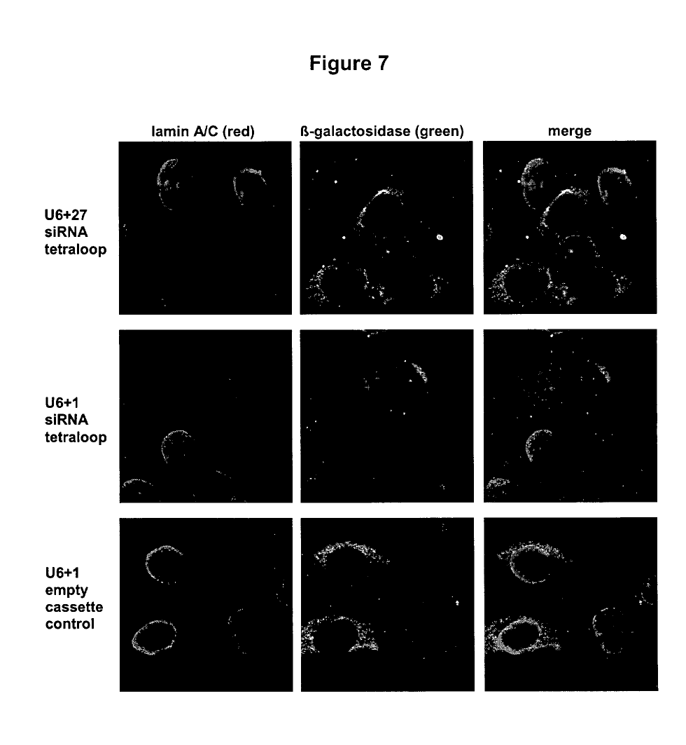

3. Inhibition of gene expression by anti-lamin A/C hairpin siRNA. Lamin A/C expression is inhibited after transfection of cells with U6 expression cassettes which encode anti-lamin siRNA. Plasmids containing either the empty U6+lgene promoter expression cassette (no RNA insert), the U6+1 gene expression cassette expressing anti-lamin AC siRNA, or the U6+27 gene promoter expression cassette expressing anti-lamin A/C siRNA were transfected into HeLa cells, along with a separate plasmid encoding β-galactosidase. The β-galactosidase plasmid is a reporter for cell transfection, as cells transfected with one plasmid are presumed to be transfected with both; thus, cells expressing β-galactosidase are presumed to be transfected with both plasmids. After antibody staining, the cells were examined by fluorescence microscopy. Cells stained with antibodies to lamin A/C fluoresced red (Santa Cruz Biotechnology primary), while those stained with antibody to β-galactosidase fluoresced green (Molecular Probes primary). The results are shown in FIG. 7.

For the empty U6+1 control expression cassette, cells fluoresced only red, indicating staining for lamin A/C only (not transfected), or fluoresced both green and red, indicating staining for both lamin A/C and β-galactosidase (indicating transfection with β-galactosidase only). For the U6+1 and U6+27 expression cassettes containing genes encoding anti-lamin A/C siRNA, cells generally fluoresced either red only (which indicates that they were expressing lamin A/C and were thus untransfected) or green only (which indicates that they were expressing β-galactosidase, but not expressing lamin A/C, indicating that they were co-transfected with both plasmids). These results show that cells that were successfully transfected with DNA (as seen by β-galactosidase expression) and an anti lamin A/C siRNA-expressing construct were deficient in lamin A/C. The U6+1 expression cassette without an anti-lamin siRNA has no detectable affect on lamin A/C expression.

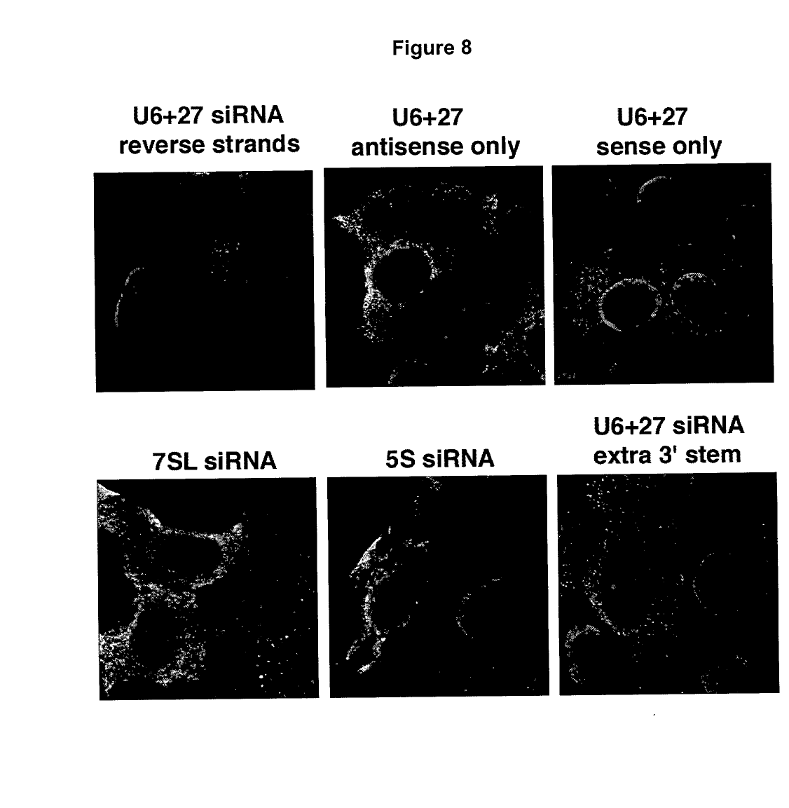

Several additional experiments were undertaken with different siRNA variants inserted in a U6+27 expression cassette. In one set of control experiments, each strand of the siRNA is expressed separately (the expected transcripts are shown in FIG. 3). The results, shown in FIG. 8, demonstrate that transfection of cultured human cells with either variant siRNA in an expression cassette had little to no effect on lamin A/C expression. In other control experiments, the two strands of the variant anti-lamin siRNA were reversed, so that the sense strand was followed by the UUUU termination sequence, while the antisense strand was followed by the tetraloop structure and no free 3′ end (the expected transcript is shown in FIG. 3). The results, as shown in FIG. 8, indicate that although there was some reduction of the lamin signal with this construct, transfection of cultured human cells with the strand switched variant siRNAin an expression cassette was not as effective as the original orientation, with the antisense strand having the 3′ overhang. Moreover, a less structured seven-nucleotide loop was tested in the siRNA transcript as well; this construct was designed to increase the chances of a nucleolytic cleavage between the strands. However, this siRNA was less effective than the siRNA with a tetraloop.

Additional experiments were undertaken to examine the effects of the overhang from the siRNA. A single nucleotide overhang from the 3′ end of the antisense strand of the siRNA was quite effective; however, a single overhang from the 3′ sense strand of the siRNA was less effective. Finally, as noted above, U6+27/siRNA construct in was made in which two U residues were removed from the sequence immediately following the siRNA duplex, causing the RNA polymerase III to transcribe through the cassette-encoded extra stem and then terminate. Although this construct also worked poorly to suppress lamin A/C, some reduction in lamin signal in transfected cells compared to non-transfected cells was consistently observed.

Moreover, the siRNA transcripts made from the U6+1 and the U6+27 expression cassettes were retained in the nuclei, whereas those from the 5S and 7SL expression cassettes were localized in the cytoplasm (as described above). This indicates that the 7SL transcripts do not also need the 3′ 7SL sequences past the siRNA insert to complete the stem.

4. Intracellular localization affects siRNA effectiveness. As noted above, anti-lamin hairpin siRNA strongly reduces lamin A/C levels in HeLa cells when expressed from the U6+27 or U6+1 cassettes. The 19-base lamin sense and antisense sequences that make up the duplex in this insert target the region of the lamin MRNA previously shown by Tuschl and coworkers (Elbashir, S. M. et al. (2001) Nature. 411: 494-498), and as shown above, to be sensitive to synthetic siRNA. The synthetic siRNA reduced lamin levels in the cell when transfected into cells using lipid-based reagents. Hairpin siRNA inhibition of lamin A/C levels appeared to use an siRNA-like mechanism, since neither the sense nor the antisense strands alone suppressed lamin levels. The effectiveness of the nuclear siRNA expression was intriguing, since there were suggestions that RNA interference might operate in the cytoplasm (Bernstein, E. et al. (2001). RNA. 7: 1509-1521). It was theoretically possible that small amounts of siRNA expressed from the U6 promoter were leaking into the cytoplasm, and that localizing larger amounts of siRNA in the cytoplasm would be even more effective. Subsequent experiments were therefore undertaken to compare the effects of U6-driven anti-lamin siRNA and to that of the same insert expressed from either the 5S or the 7SL cassette.

Plasmids carrying the cassettes for expressing anti-lamin A/C hairpin siRNAs were cotransfected into HeLa cells along with a plasmid coding for β-galactosidase to mark transfected cells. Lamin (red) and β-galactosidase (green) were detected in fixed cells by Immunolocalization. The nuclei of the untransfected cells exhibit a strong red fluorescent signal as detected by antibodies to lamin A/C. The level of lamin A/C in the nuclei of cells transfected with the U6 cassettes was severely reduced, but a decrease in red signal was not observed when the cells were transfected with the 5S or 7SL cassettes expressing anti-lamin siRNA. These results demonstrate that expression of the same anti-lamin siRNA hairpin that causes a reduction in lamin accumulation when expressed from the U6 promoter does not reduce lamin levels when expressed from the 5S and 7SL promoters.

Thus, 5S- and 7SL-driven expression of the insert did not reduce lamin A/C levels. These results demonstrate that the promoter cassette and resulting subcellular location of the product RNA are important considerations in designing effective strategies for expression of therapeutic RNAs.

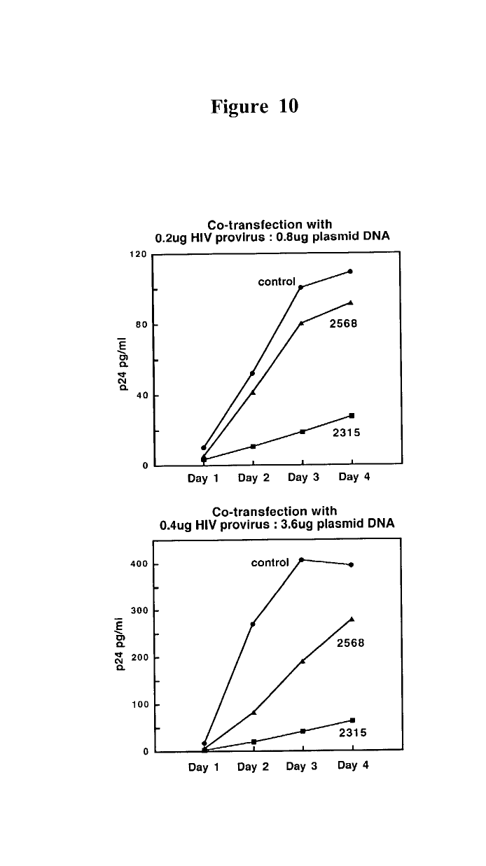

5. Inhibition of gene expression by anti-HIV siRNAs. Intracellular expression of effective siRNA is useful in experimentally ablating cellular gene products, and is also contemplated to be extremely useful as gene therapy against viral infection. As an initial demonstration of this utility, the effectiveness of a hairpin siRNA expressed from a U6 expression cassette (U6+27) targeted against gene expression from co-transfected HIV-1 provirus was examined. Hairpin siRNAs, designed to target sequences within the HIV-1 pol sequence (FIG. 9), were expressed from the U6+27 cassette and tested for their ability to interfere with gene expression from the long HIV transcript. Two examples (positions 2315 and 2568 in the HIV NL4-3 sequence, Genbank accession #AF070521) were compared to control cassettes containing no inserts (FIG. 10). (Position 2315 refers to the sequence from position 2315-2333, and position 2568 refers to the sequence from position 2568-2586).

In this experiment, HIV-1 polymerase mRNA was targeted using hairpin siRNAs expressed from the U6+27 cassette. HIV-1 gene expression following cotransfection of cells with provirus was measured using an immunoassay for HIV p24, as described above. The siRNA inserts were directed against positions 2315 and 2568 in the HIV-1 RNA sequence. Their ability to interfere with HIV- 1 gene expression was compared to that of a control U6+27 cassette with no insert (FIG. 10).

The results demonstrated that construct 2315 consistently inhibited HIV gene expression and, while there was significant variability between experiments, immunoassay for p24 showed that viral gene expression was decreased by up to 90%. In contrast, the second insert targeted to a different sequence in the same region, position 2568, did not consistently reduce p24 levels by greater than 50%. Thus, different target sequences for siRNA inhibition are not equally effective, just as synthetic siRNA sequences vary dramatically in their effects against the same target (Elbashir, S. M. et al. (2001) Nature. 411: 494-498; Elbashir, S. M. et al (2001) Genes Dev. 15: 188-200). Based upon these results, it is contemplated that several sequences against any gene of interest should be examined when expressing these RNAs within the cell, in order to determine which sequences are most effective.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the following claims.

What is claimed is:

1. A composition comprising an siRNA molecule, wherein the molecule comprises a first region, a second region, and a third region, wherein the first region is complementary to and paired to the second region forming a double stranded region such that the double stranded region comprises about 18 to about 25 nucleotide pairs, and wherein the third region links said first region to said second region.

2. The composition of claim 1, wherein the first region or the second region of the siRNA molecule is complementary to a region of a target RNA molecule.

3. The composition of claim 2, wherein the siRNA molecule further comprises at least a fourth region, wherein the fourth region enhances the function of the siRNA.

4. The composition of claim 3, wherein the fourth region of the siRNA comprises a cellular destination signal.

5. An expression cassette comprising a promoter operably linked to a gene encoding an siRNA molecule, wherein the siRNA molecule comprises a first region and a second region, wherein the first region is complementary to and paired to the second region forming a double stranded region such that the double stranded region comprises about 18 to about 25 nucleotide pairs.

6. The expression cassette of claim 5, wherein the siRNA molecule further comprises a third region, wherein the third region links the first region to the second region.

7. The expression cassette of claim 5, wherein the first region or the second region of the siRNA molecule is complementary to a region of a target RNA molecule.

8. The expression cassette of claim 6, wherein the siRNA molecule further comprises at least a fourth region, wherein the fourth region enhances the function of the siRNA.

9. The expression cassette of claim 8, wherein the fourth region of the siRNA molecule comprises a cellular destination signal.

10. The expression cassette of claim 5, wherein the promoter is a polymerase III promoter.

11. The expression cassette of claim 10, wherein the promoter is a promoter from a U6 snRNA gene, from a 5S rRNA gene, or from a 7SL signal recognition particle RNA gene.

12. The expression cassette of claim 5, wherein the promoter is modified to possess different specificity.

13. A vector comprising at least one expression cassette of claim 4 and wherein the vector is a mammalian cell vector.

14. The vector of claim 13, wherein the vector comprises at least one additional second gene operably linked to a second promoter and wherein the second gene is selected from the group consisting of marker genes, reporter genes, and selectable genes.

15. A mammalian cell expressing an siRNA molecule of claim 1.

16. A mammalian cell expressing an siRNA molecule of claim 2.

17. A mammalian cell expressing an siRNA molecule of claim 3.

18. A mammalian cell expressing an siRNA molecule of claim 4.

19. A mammalian cell transfected with an expression cassette of claim 5.

20. A mammalian cell transfected with an expression cassette of claim 6.

21. A mammalian cell transfected with an expression cassette of claim 7.

22. A mammalian cell transfected with an expression cassette of claim 8.

23. A mammalian cell transfected with an expression cassette of claim 9.

24. A mammalian cell transfected with an expression cassette of claim 10.

25. A mammalian cell transfected with an expression cassette of claim 11.

26. A mammalian cell transfected with a vector of claim 13.

27. A method of transfecting a mammalian cell, comprising:
a) providing a mammalian cell and an expression cassette according to claim 5; and
b) transfecting the cell with the cassette.

28. A method of expressing siRNA in a mammalian cell, comprising:
a) providing a mammalian cell transfected with an expression cassette of claim 5; and
b) growing said cell under conditions such that the siRNA is expressed.

29. A method of expressing siRNA in a mammalian cell, comprising:
a) providing a mammalian cell and an expression cassette of claim 5; and
b) transfecting the cell with the cassette under conditions such that the cassette is expressed.

30. A method of decreasing expression of a gene in a mammalian cell, comprising:
a) providing a cell transfected with an expression cassette of claim 5, where the siRNA targets a gene in the cell; and
b) growing the cell under conditions such that the cassette is expressed, thereby decreasing expression of the gene in the mammalian cell.

31. A method of decreasing expression of a gene in a mammalian cell, comprising:
a) providing a mammalian cell and an expression cassette according to claim 5, where the siRNA targets a gene in the cell; and
b) transfecting the cell with the cassette under conditions such that the cassette is expressed,
thereby decreasing expression of the gene in the mammalian cell.
